(19) United States
(12) Patent Application Publication
(10) Pub. No.: US 2026/0027155 A1
(43) Pub. Date: Jan. 29, 2026

(54) COMPOSITIONS AND METHODS RELATED TO POTENT CYTOTOXIC M-CENK CELLS FROM CD3/CD14-DEPLETED APHERESIS PRODUCTS

(71) Applicant: ImmunityBio, Inc., San Diego, CA (US)

(72) Inventors: Ferdous Anower-E-Khuda, San Diego, CA (US); Gene Yeh, San Diego, CA (US); Syed Raza Ali, San Diego, CA (US); Manju Saxena, San Diego, CA (US); Leonard Sender, Laguna Beach, CA (US); Patrick Soon-Shiong, Los Angeles, CA (US)

(21) Appl. No.: 19/277,274

(22) Filed: Jul. 22, 2025

Related U.S. Application Data
(60) Provisional application No. 63/674,702, filed on Jul. 23, 2024.
(60) Provisional application No. 63/682,088, filed on Aug. 12, 2024.

(51) Int. Cl. A61K 35/17; A61K 40/15; A61K 40/42; A61P 35/00; C07K 14/705; C07K 16/28; C12N 5/00; C12N 5/0783

(52) U.S. Cl. CPC A61K 35/17; A61K 40/15; A61K 40/421; A61P 35/00; C07K 14/70503; C07K 16/2887; C12N 5/0018; C12N 5/0646; C12N 2501/2312; C12N 2501/2315; C12N 2501/2318

(57) ABSTRACT

Provided herein are compositions and methods related to potent cytotoxic M-CENK cells from CD3/CD14 depleted apheresis products.

### RELATED APPLICATIONS

This application claims priority to 63/674,702, filed on Jul. 23, 2024, and 63/682,088, filed on Aug. 12, 2024. The entire content of each of the aforementioned provisional applications is herein incorporated by reference for all purposes.

### BACKGROUND

Natural killer (NK) cells kill cancer cells through one of the following mechanisms: (1) releasing perforin and granzyme to cause apoptosis of malignant cells; (2) generating cytokines like interferon-gamma (IFN-γ) that exert anti-tumor effects through different mechanisms; and (3) triggering antibody-dependent cell-mediated cytotoxicity (ADCC) of the tumor cells. The latter mechanism depends on the presence of the CD16 receptor on NK cells.

Cytokine-enhanced memory-like NK (M-CENK, aka., m-ceNK) cells represent a promising approach in NK Cell therapeutics. However, M-CENK cells express CD16 at levels that may limit their ability to induce tumor killing via the ADCC mechanism. CD16 expression is not stable in the cytokine-induced expansion/activation of NK cells from apheresis products created by previously described methods, and hence enriched NK cells cannot be effectively used in ADCC without improvements in CD16 expression and/or stabilization. There is a need for M-CENK cells with increased CD16 expression and/or CD16 stabilization to improve their ADCC effects accordingly.

Additionally, there is also a need to reduce the time that it takes to produce M-CENK cells for the timely treatment of cancer patients.

### SUMMARY

In some embodiments, this disclosure provides a method of generating memory-like cytokine enhanced natural killer (M-CENK) cells from a blood sample, the method comprising: removing CD3+ cells and CD14+ cells from the blood sample to obtain a CD3− CD14− population of cells; incubating the CD3− CD14− population of cells with a first cytokine composition comprising a stabilized IL-15 analog or fusion protein comprised thereof for a first period, and incubating the CD3− CD14− population of cells with a second cytokine composition comprising i) IL-15 or a stabilized derivative thereof, ii) IL-18 or a stabilized derivative thereof, and iii) IL-12 or a stabilized derivative thereof; and continuing to incubate the CD3− CD14− fraction cells with the second cytokine composition for a second period, thereby producing a population of CD56+ enriched m-ceNK cells. In some embodiments, the IL-15 or stabilized derivative thereof is in a concentration ranging from 25 to 175 ng/ml. In some embodiments, the IL-18 or stabilized derivative thereof is in a concentration ranging from 25 to 50 ng/mL. In some embodiments, the IL-12 or stabilized derivative thereof is in a concentration ranging from 5 to 10 ng/mL. In some embodiments, the blood sample is obtained from a cancer patient. In some embodiments, the cancer patient is a small cell lung cancer, an ovarian cancer, a breast cancer, a leukemia, a myelogenous leukemia, or an acute lymphoblastic leukemia. In some embodiments, removing CD3+ cells and CD14+ cells from the blood sample to obtain a CD3− CD14− population of cellsis by magnetic cell separation. In some embodiments, the first period lasts 7 to 12 days. In some embodiments, the second period has a length of 14-16 hours.

In some embodiments, this disclosure provides the population of CD56+ enriched cells produced from any one of the methods disclosed above. In some embodiments, greater than 90%, greater than 92%, greater than 93%, greater than 94%, greater than 95%, greater than 96%, greater than 97%, greater than 98%, greater than 99% of the cells are CD56+. In some embodiments, greater than 60%, greater than 61%, greater than 62%, greater than 63%, greater than 64%, greater than 65%, greater than 68%, greater than 70%, greater than 72%, greater than 75%, greater than 76%, greater than 77%, greater than 78%, or greater than 80% of the cells are CD16+. In some embodiments, this disclosure provides a method of treating a patient diagnosed with cancer, the method comprising: obtaining a blood sample from the patient, removing CD3+ cells and CD14+ cells from the blood sample to obtain a CD3− CD14− fraction, contacting the CD3− CD14− fraction of cells with IL-15 or a stabilized derivative thereof for a first period of time, contacting the CD3− CD14− fraction of cells with IL-12 or a derivative thereof, IL-18 or a derivative thereof, and IL-15 or a derivative thereof, for a second period of time, thereby obtaining a population of CD56+ enriched M-CENK cells. contrifuging the cell culture to produce a supernant; isolating exosomes from the supernatant; and (g) administering the isolated exosomes to the patient, thereby treating the cancer. Also provided herein is a method of treating a patient diagnosed with cancer, the method comprising administering a therapeutically effective amount of the CD56+ enriched M-CENK cells disclosed above to the patient. In some embodiments, the cancer is a solid tumor. In some embodiments, the cancer is a metastatic solid tumor. In some embodiments, the cancer is a small cell lung cancer, an ovarian cancer, a breast cancer, a leukemia, a myelogenous leukemia, or an acute lymphoblastic leukemia. In some embodiments, greater than 90% of the CD56+ enriched population of cells are CD56+. In some embodiments, greater than 68% of the CD56+ enriched population of cells are CD16+. In some embodiments, the method further comprises administering an antibody. In some embodiments, the antibody is specific for a tumor antigen. In some embodiments, the antibody is an anti-CD20 antibody. In some embodiments, the antibody is a checkpoint inhibitor antibody. In some embodiments, the antibody is an anti-PD-1, an anti-PD-L1, or an anti-CTLA-4 antibody. In some embodiments, this disclosure provides a kit for preparing CD56+M-CENK cells, comprising: reagents for depleting CD3+ cells and CD14+ cells from a blood sample; and IL-15 or N-803, IL-12, and IL-18.

In some embodiments, this disclosure provides a kit for preparing CD56+M-CENK cell exosomes, the kit comprising: i) reagents for depleting CD3+ cells and CD14+ cells from a blood sample; ii) IL-15 or a fusion protein comprised thereof, iii) IL-12 or a fusion protein comprised thereof, and iv) IL-18 or a fusion protein comprised thereof.

In some embodiments, this disclosure provides a population of CD56+ enriched M-CENK cells wherein at least 90% of the cells are CD3−, CD14−, and CD56+, and wherein at least 60% of the cells express CD16.

In some embodiments, the population of CD56+ enriched M-CENK cells obtained using as described above are freezed and thawed.

In some embodiments, this disclosure provides a cell culture mixture comprising a population of blood cells that are CD3− CD14−, a first cytokine composition comprising a stabilized IL-15 analog or fusion protein. In some embodiments, the cell culture mixture further comprises a second cytokine composition comprising one or more of i) IL-15 or a stabilized derivative thereof, ii) IL-18 or a stabilized derivative thereof, and iii) IL-12 or a stabilized derivative thereof.

The foregoing general description and the following detailed description are exemplary and explanatory and are intended to provide further explanation of the disclosure. Other objects, advantages, and novel features will be readily apparent to those skilled in the art.

### BRIEF DESCRIPTION OF THE DRAWING

The objects, features, and advantages will be more readily appreciated upon reference to the following disclosure when considered in conjunction with the accompanying drawings.

FIG. 1 is a schematic representation of a method to produce M-CENK cells with the method described in previous disclosure (bottom flow chart) and a method to create M-CENK cells from a CD3− CD14− fraction from apheresis product.

FIG. 2A shows results of intracellular IFN-γ staining of the thawed M-CENK cells produced using the methods disclosed herein. FIG. 2B shows results of cytotoxicity of the thawed M-CENK cells.

FIG. 3 compares the cell culture expansion kinetics for cells expanded from cryopreserved Apheresis Material Intermediate (AMI) vs CD3− CD14− fraction of the same fresh apheresis product isolated using the CliniMACS® Plus Process.

FIG. 4 shows plots of a flow cytometry-based analysis of CD16 Expression on M-CENK cells: M-CENK cells generated from Apheresis product vs. CD3− CD14− fraction of the Apheresis were stained with the indicated antibodies and analyzed on a Flow cytometer for receptor expression abundance. Increased CD16 expression (>60%) was observed on M-CENK cells generated from CD3− CD14− fraction of the Aphersis product as compared to Apheresis product derived M-CENK cells (<10% CD16 expression). A higher percentage of CD16+ cells in the population correlates to a higher CD16 expression in the population.

FIG. 5 shows flow cytometry-based analysis of IFN-gamma Expression on M-CENK cells: M-CENK cells generated from Apheresis Product or from the CD3− CD14− fraction of the Apheresis were stained with indicated antibodies and analyzed on a Flow cytometer. Results show IFN-gamma histogram, blue (left curves): isotype control and red (right curves): test sample.

FIG. 6 shows plots representing a comparison of cytotoxicity of M-CENK Cells against K562 cells: cytotoxicity of M-CENK cells from two sources against K562 target cells. Cytotoxicity is expressed as % target cell lysis as evaluated by calcein release assay.

FIG. 7 shows plots of M-CENK cell activity against NK-resistant tumor cells: cytotoxicity of M-CENK cells from two sources against the indicated tumor cells. Cytotoxicity is expressed as % target cell lysis as evaluated by calcein release assay.

FIG. 8 is an ADCC plot of M-CENK Cells derived from CD3− CD14− Fraction of the Apheresis against TMD-5 Cells: M-CENK cells were mixed with TMD-5 in the presence/absence of Rituxan® antibody (i.e., an anti-CD20 antibody). ADCC is expressed as % target cell lysis in the presence of Rituxan® antibody, as evaluated by calcein release assay. ADCC was calculated after 3 hours of reaction time.

FIG. 9 is a plot showing a comparison of ADCC of M-CENK cells derived from Apheresis Product vs M-CENK derived from CD3− CD14− fraction of the Apheresis against TMD-5 cells. M-CENK cells were mixed with TMD-5 in the presence of the Rituxan® antibody. ADCC is expressed as % target cell lysis in the presence of the Rituxan® antibody, as evaluated by calcein release assay. ADCC was calculated after 3 hours of reaction time.

### DETAILED DESCRIPTION

### Overview

Described herein are improvements to existing apheresis methodology that deplete the apheresis product of CD3⁺ and CD14⁺ cells, creating a CD3⁻/CD14⁻ double-depleted apheresis product (i.e., an apheresis product where the CD3− expressing and CD14− expressing fractions of cells have been removed from the apheresis product). The inventors have developed this double-depleted methodology to produce a different population of immune cells compared to previous disclosure (traditional) apheresis methodology, in particular, one that is enriched in B-cells, NK cells, dendritic cells, and granulocytes compared to the traditional apheresis product. The inventors have further found that from the population of immune cells produced by double-depleted methods as described herein, M-CENK cells can be created with higher CD16 expression compared to traditional methods and enriched CD56 expression compared to traditional methods that provide conventional M-CENK cells. These M-CENK cells created from a double-depleted apheresis product can exhibit enhanced functional potency, in particular, greater cytotoxicity against targets of interest (in particular by ADCC mechanisms). These cells also can be expanded for therapeutic use and administered to a patient in as few as 10-12 days after a blood draw from the patient. The M-CENK cells described herein can be used to target a broad range of target cells, for example, cells from a variety of cancers.

The disclosure also provides compositions and kits comprising a plurality of M-CENK cells created from the CD3⁻/CD14⁻ double-depleted apheresis product. Methods of producing M-CENK cells and using the M-CENK cells to treat cancer are also provided. This application incorporates the disclosure of U.S. Pat. No. 10,258,649 by reference in its entirety.

### Terminology

Unless defined otherwise, all technical and scientific terms used herein have the same meaning as commonly understood by one of ordinary skill in the art.

In this specification and in the claims that follow, reference will be made to a number of terms that shall be defined to have the following meanings:

The terminology used herein is for the purpose of describing particular embodiments only and is not intended to be limiting. As used herein, the singular forms “a,” “an,” and “the” are intended to include the plural forms as well, unless the context clearly indicates otherwise. Thus, for example, reference to “a natural killer cell” includes a plurality of natural killer cells.

All numerical designations, e.g., pH, temperature, time, concentration, amounts, and molecular weight, including ranges, are approximations that are varied (+) or (−) by increments of 0.1 or 1.0, where appropriate. It is to be understood, although not always explicitly stated, that all numerical designations may be preceded by the term “about.”

As used herein, “+”, when used to indicate the presence of a particular cellular marker, means that the cellular marker is detectably present in fluorescence activated cell sorting over an isotype control; or is detectable above background in quantitative or semi-quantitative RT-PCR.

As used herein, “−”, when used to indicate the presence of a particular cellular marker, means that the cellular marker is not detectably present in fluorescence activated cell sorting over an isotype control; or is not detectable above background in quantitative or semi-quantitative RT-PCR.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

It is also to be understood, although not always explicitly stated, that the reagents described herein are merely exemplary and that equivalents of such are known in the art.

“Optional” or “optionally” means that the subsequently described event or circumstance can or cannot occur, and that the description includes instances where the event or circumstance occurs and instances where it does not.

The term “comprising” is intended to mean that the compositions and methods include the recited elements but not excluding others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claims. “Consisting of” shall mean excluding more than trace amount of other ingredients and substantial method steps. Embodiments defined by each of these transition terms are within the scope of the disclosure.

As used herein, the terms “cytotoxic” and “cytolytic”, when used to describe the activity of effector cells such as NK cells, are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell. Without wishing to be bound by theory, it is believed that the cytotoxic effect of NK cells is due to cytolysis.

As used herein, the term “natural cytotoxicity” refers to the function of Natural killer (NK) cell to kill variety of target cells, including tumor cells, virus-infected cells, without prior activation by an antigen on these cells. NK cell has variety of natural cytotoxicity receptors and when these receptors encounter specific ligands on target cells, they trigger the release of cytotoxic granules which induce cell death in the target cell. See, Vivier, E., et al., Science. 2011 Jan. 7; 331(6013): 44-49; Hudspeth, K., et al., Front Immunol. 2013 Mar. 20; 4:69; Barrow, A. D. et. al., Frontiers in Immunology, May 2019, Vol 10, Article 909.

The term “kill” with respect to a cell/cell population is directed to include any type of manipulation that will lead to the death of that cell/cell population.

The term “cytokine” or “cytokines” refers to the general class of biological molecules which effect cells of the immune system. Exemplary cytokines include but are not limited to FLT3 ligand, interferons, and interleukins (IL), in particular IL-2, IL-12, IL-15, IL-18 and IL-21.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. The term “administering” or “administration” of a monoclonal antibody or a natural killer cell to a subject includes any route of introducing or delivering the antibody or cells to perform the intended function. Administration can be carried out by any route suitable for the delivery of the cells or monoclonal antibody. Thus, delivery routes can include intravenous, intramuscular, intraperitoneal, or subcutaneous delivery. In some embodiments the M-CENK cells are administered directly to the tumor, e.g., by injection into the tumor. In some embodiments the M-CENK cells described herein are administered parenterally, e.g., by injection, infusion or implantation (subcutaneous, intravenous, intramuscular, intravesicularly, or intraperitoneal).

The term “expression” refers to the production of a gene product.

As used herein, the terms “cytotoxic” when used to describe the activity of effector cells such as NK cells, relates to killing of target cells by various biological, biochemical, or biophysical mechanisms.

The terms “decrease,” “reduced,” “reduction,” and “decreased” are all used herein to refer to a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

The term “cancer” refers to all types of cancer, neoplasm, or malignant tumors found in mammals, including leukemia, carcinomas, and sarcomas. Exemplary cancers include cancer of the brain, breast, cervix, colon, head & neck, liver, kidney, lung, non-small cell lung, melanoma, mesothelioma, ovary, sarcoma, stomach, uterus, and medulloblastoma. Additional examples include Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

The term “therapeutically effective amount” or “effective amount” refers to the amount required to ameliorate the symptoms of a disease relative to an untreated patient. The effective amount of active compound(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Numerical quantities given herein are approximate unless stated otherwise, meaning that the term “about” or “approximately” can be inferred when not expressly stated. When “about” is applied to the beginning of a numerical range, it applies to both ends of the range.

“NK cell”, as referred to herein is a type of immune cell that has granules (small particles) with enzymes that can kill tumor cells and/or cells infected with a virus. It is a type of white blood cell and one skilled in immunology is aware of what an NK cell is. As used herein, NK cell, may refer to any natural killer cell. In certain embodiments, the NK cell may be an aNK cell (also known as an NK-92 cell), a haNK cell, a T-haNK cell, a primary NK cell, a KHYG-1 cell, or a variant or derivative thereof. In some embodiments, the NK cell can be genetically engineered and in other embodiments it cannot be genetically engineered. In some embodiments, the NK cell does not express Tor B cell receptors. In some embodiments, the NK cell may be recognized by absence of CD3. The NK cell may additionally or alternatively be classified as CD56bright or CD56dim_In some embodiments, the NK cell may be a memory-like cytokine enhanced (m-CENK) cell. In some embodiments, the “NK cell” may be referring to a cell line.

NK-92 is an NK-like cell line that was initially isolated from the blood of a subject suffering from a large granular lymphoma and subsequently propagated in cell culture. The NK-92 cell line has been described (see e.g., Klingemann & al. (2016) Front Immunol 7:91). NK-92 cells have a CD3−/CD56+ phenotype that is characteristic of NK cells. They express all of the known NK cell-activating receptors except CD 16, but lack all of the known NK cell inhibitory receptors except NKG2A/CD94 and IL T2/LIR1, which are expressed at low levels. Furthermore, NK-92 is a clonal cell line that, unlike the polyclonal NK cells isolated from blood, expresses these receptors in a consistent manner with respect to both type and cell surface concentration. Similarly, NK-92 cells are not immunogenic and do not elicit an immune rejection response when administered therapeutically to a human subject. NK-92 cells are well tolerated in humans with no known detrimental effects on normal tissues.

Titles or subtitles may be used in the specification for the convenience of a reader, which are not intended to influence the scope of the present disclosure. Additionally, some terms used in this specification are more specifically defined below.

### Double-Depleted Apheresis Products

Described herein is an apheresis product (also referred to herein as “apheresis material”) derived from a subject (e.g., a newly diagnosed cancer patient) that is depleted of CD3+ and CD14+ cells (thereby creating a double-depleted apheresis product lacking cells expressing CD3 and CD14) using a cell separation technique (for example, a magnetic cell separation platform or fluorescence cell sorting platform).

Cells can be expanded in culture vessels in the presence of IL-15 or N-803 for a period of time (e.g., 7-12 days, 7-11 days, 7-10 days, 7-9 days, 7-8 days, 8-12 days, 9-12 days, 10-12 days, or 11-12 days, thereby creating cytokine-enhanced natural killer cells (CENK cells)) and then stimulated with a cytokine cocktail (IL-15/N-803)/IL-18/IL-12) for a period of time (e.g., 14-16 hours, 14-15 hours, or 15-16 hours) to generate memory cytokine-enhanced natural killer cells (M-CENK cells). Cells are then ready for therapeutic administration (fresh or cryopreserved) or cryopreservation.

M-CENK cells described herein are produced through cytokine induction of cells derived from remaining double-depleted CD3−/CD14− apheresis product. In some instances, they are produced from a CD3−/CD14− double-depleted apheresis product fraction through cytokine induction with a cytokine cocktail. In some instances, they are produced during the process of enriching cytokine-enhanced natural killer cells (CENK cells) by culturing apheresis fractions as described in previous disclosure of Potent M-CENK (US 2024/0132844A1) in a growth media in the presence of IL-15, or a stabilized derivative thereof. For purpose of this disclosure, a stabilized derivative of an IL-15 refers to an IL-15 polypeptide that is engineered to possess exhibit enhanced stability under various conditions, including pharmacokinetic conditions, making them more useful for therapeutic, industrial, and research applications. Nonlimiting examples of such stabilized derivatives include an IL-15 analog, an IL-15 superagonist, IL-15: IL15RαSu, or fusion protein thereof. Fusion proteins derived from IL-15 or IL-15: IL15RαSu comprise an Fc domain, IgG, and/or tissue factor conjugates, wherein the IL-15 or IL-15: IL15RαSu is genetically fused or covalently linked to the stabilizing protein. Following expansion in the presence of IL-15, an IL-15 analog, an IL-15 superagonist, or fusion protein thereof and a growth media, CENK cells are then cultured in a growth media containing a cytokine cocktail (as described above) to produce M-CENK cells. M-CENK cells can then be administered to a subject fresh or cryopreserved.

M-CENK cells produced by the methods disclosed herein differ from the previously described M-CENK cells produced as described in previous disclosure of Potent M-CENK (US Pat. Pub. No. 2024/0132844A1) (i.e., the methods that did not deplete an apheresis product of any cells expressing a particular marker, for example, CD3 and/or CD14) in several aspects. Phenotypically, they exhibit higher CD16 expression levels or CD16 activity and have improved cytotoxic abilities compared to M-CENK cells produced from non-CD3−/CD14− double-depleted apheresis fractions.

Thus, in terms of phenotypes, the M-CENK cells provided herein typically have high expression levels of CD56 (e.g., >90%) and CD16, in particular, higher expression levels (e.g., >30%) than conventional M-CENK cells created by non-double-depleted methodology (i.e., created by method that did not remove the fractions of cells expressing CD3 and CD14).

Method of Generation, Isolating, and Culturing M-CENK Cells from Double-Depleted (CD3− CD14−) Apheresis Products

Natural killer (NK) cells are cytotoxic innate lymphoid cells emerging as acellular immunotherapy for various malignancies. NK cells depend on interleukin-15 (IL-15) for their survival, proliferation, and cytotoxic function. NK cells differentiate into memory-like cells (i.e., M-CENK cells) with enhanced effector function after a brief activation with IL-12, IL-15, and IL-18. Described herein is the creation of M-CENK cells from a double-depleted (CD3− CD14−) apheresis product.

### Collecting Blood and Apheresis Products

Apheresis products can be collected from a subject (i.e., a subject having or suspected of having cancer, for example, a metastatic or solid metastatic tumor) or a subject without cancer. Blood can be taken from a subject (e.g., one having or suspected of having a cancer, for example, a metastatic cancer or metastatic solid tumor) and subjected to apheresis. Apheresis products can be collected, for example, by a continuous mononuclear cell collection.

The subject can be a mammalian subject having or suspected of having cancer. The subject can be a subject that does not have cancer. For example, the cells from the apheresis product can be autologous (i.e., cells from the blood of the same subject to which the M-CENK cells will then be administered) or allogenic [i.e., cells taken from a different individual (Sibling or a parent) related to the subject, for example, a human that does not have and is not suspected of having cancer].

CD3−/CD14− cells (i.e., an apheresis product from which CD3+ and CD14+ cells have been removed, also referred to as a “double-depleted (CD3− CD14−) apheresis product”) can be isolated from an apheresis product by methods well known in the art, for example, incubating magnetic beads coupled with antibody against CD3 and CD14 with the apheresis product so that the CD3 and CD14 cells will bind to the magnetic beads, and subsequently isolating the CD3 and CD14 cells that are bound to the beads in presence of a magnetic field to create a fraction that is void of CD3 and CD14 cells.

Briefly, following the collection of an apheresis product, the apheresis product can be subject to buffer exchange, for example, to exchange to a PBS/EDTA/HA buffer, for example. The apheresis product can then be subject to treatment to prevent non-specific antibody binding, with the treatment of, for example, intravenous immunoglobin (IVIG) or an Fc receptor block or human albumin. Briefly, the Apheresis product can be collected from donors/patient as continuous mononuclear cell (MNC) collection using the Spectra Optia® Apheresis System. The fresh apheresis bags are then transferred to the GMP lab for cell processing and testing.

Following treatment with the non-specific antibody binding blocker, cells can then be treated with an anti-CD3 reagent (e.g., magnetic beads coated with anti-CD3 antibodies) from Miltenyi, followed by a wash, centrifugation, and resuspension in an aqueous solution. Following resuspension, the solution can be passed through a filter to remove cell aggregates and other unwanted debris. The remaining cellular solution can then be subject to additional separation techniques that remove CD3+ cells. This process can be repeated for the CD3− fraction using an anti-CD14 reagent (e.g., magnetic beads coated with anti-CD14 antibodies) and removing CD14+ cells from the CD3− fraction. Suitable cell separations known in the art can be utilized, for example, with the CliniMACS® platform and methods. It is understood that the process could be reversed, whereby CD14+ cells are removed prior to the CD3+ cells. In embodiments, the cell separation platform employed or otherwise utilizes the CliniMACS® platform and technology from Miltenyi Biotec or equivalent.

Expanding and Enriching CD56+NK Cells from the CD3−/CD14− Double-Depleted Apheresis Product

The isolated enriched CD56+ cells from the double-depleted (CD3− CD14−) apheresis product described above can be expanded in a suitable growth medium in the presence of a cytokine (e.g., IL-15 or N-803) to create cytokine-enhanced natural killer cells (CENK cells). Expanding refers to growing an isolated population of target cells so that the target cells increase in number, providing a sufficient number of cells to provide for multiple administrations of M-CENK cells from a single blood draw, i.e., providing enough cells to allow for about 15 to 30 administrations in the subject from a single blood draw.

In some embodiments, the growth medium is the CTS-NK Xpander media including 10% Human AB serum and CTS NK Xpander supplements plus the cytokine (e.g., IL-15 or N-803). In some embodiments, once required viable cell density is achieved, typically after culturing for 10-12 days, the cells are stimulated with the cytokine or cytokine cocktail overnight to generate M-CENK cells. These cells can be expanded in a suitable bioreactor for example, a G-REX flask from Wilson Wolf and single-use bioreactor like Xuri bioreactor (from Cytiva) for scale-up cultures. Cells can also be expanded in a suitable bioreactor, for example, a G-REX flask from Wilson Wolf Manufacture. In one illustrative embodiments, materials and methods can also be found in U.S. Pat. Nos. 11,773,364, 10,801,005, and WO 2022/187207 A1, which are incorporated by reference as if fully set forth herein. In some embodiments, the VivaBioCell platform is not utilized.

Accordingly, the disclosure provides a method of growing CENK cells obtained from a CD3−/CD14− fraction of an apheresis product (that can be used for the creation of M-CENK cells), the method comprising obtaining an apheresis product from the blood of a subject; removing CD3+ cells by cell separation; removing CD14+ cells by cell separation; and expanding the the double depleted (CD3− CD14) cells in a growth media in the presence of a cytokine (e.g., IL-15, human IL-15, stabilized IL-15, IL-15 analogs, N-803, and IL-15 fused to albumin through a Cys34 linkage, for example) for a period of time (e.g. 7-12 days). Cytokines can be used to expand the double depleted cells are described in U.S. Pat. Nos. 11,845,783 and 11,975,059.

These cells can be cultured and expanded in growth media in the presence of a cytokine (e.g., IL-15 or N-803 at a concentration or about 20 to about 300 ng/ml; about 25 to about 175 ng/ml; about 45 to about 165 ng/ml; about 55 to about 155 ng/mL; about 65 to about 145 ng/ml; about 75 to about 135 ng/mL; about 85 to about 125 ng/ml; about 95 to about 115 ng/ml; about 105 ng/ml; about 35 to about 175 ng/ml; about 45 to about 175 ng/mL; about 55 to about 175 ng/ml; about 65 to about 175 ng/ml; about 75 to about 175 ng/ml; about 85 to about 175 ng/ml; about 95 to about 175 ng/ml; about 105 to about 175 ng/ml; about 115 to about 175 ng/ml; about 125 to about 175 ng/ml; about 135 to about 175 ng/ml; about 145 to about 175 ng/mL; about 155 to about 175 ng/ml; about 165 to about 175 ng/ml; about 20 to about 165 ng/ml; about 20 to about 155 ng/mL; about 20 to about 145 ng/ml; about 20 to about 135 ng/ml; about 20 to about 125 ng/ml; about 20 to about 115 ng/mL; about 20 to about 105 ng/ml; about 20 to about 95 ng/ml; about 20 to about 85 ng/ml; about 20 to about 75 ng/ml; about 20 to about 65 ng/ml; about 20 to about 55 ng/ml; about 20 to about 45 ng/ml; about 20 to about 35 ng/ml; or about 20 to about 25 ng/ml).

The cells can be cultured and expanded in the presence of a cytokine for a period of time of about 7 to 12 or 8 to 12 or 9 to 12 or 10 to 12 or 11 to 12 or 12 days, about 7 to 9, or 8 to 9 days, or 7 to 8 days. Cells can be expanded and cultured in a suitable bioreactor.

Creating M-CENK Cells from the CD3−/CD14− Double Depleted Fraction

Stimulation of a memory phenotype of the resulting CENK cells (i.e., M-CENK can then be achieved by the addition of a natural-killer growth medium (NK-GM) containing a cytokine cocktail. Suitable cytokines that can be added to the medium for M-CENK creation may include one or more cytokines selected from the group consisting of N-803 or IL-15, IL-12, and/or IL-18.

Briefly, the expanded CENK cells of the CD3−/CD14− double-depleted apheresis product (or expanded cells from that population) can be cultured for a period of time in growth media in the presence of various cytokines (i.e., a “cytokine cocktail”) to create, expand, or otherwise enrich for memory cytokine-enhanced natural killer cells (M-CENK cells, created from the CENK cells described in the preceding section). The memory induction media can comprise a cytokine cocktail (e.g., IL-15 or a stabilized analog, superanalog, or fusion protein thereof or N803; IL-12 or a stabilized analog, superanalog, or fusion protein thereof; and IL-18 or a stabilized analog, superanalog, or fusion protein thereof). For example, the cytokine cocktail can comprise recombinant cytokines derived from human cytokine coding sequences. Creating or enriching refers to increasing the percentage of M-CENK target cells in a heterogeneous cell population comprising an enriched CD56+ NK cell population from a double-depleted apheresis product. The creation or enrichment period may be about 7 to 12 days followed by memory induction by stimulating the enriched CENK cells with the cytokine cocktail (IL-15 plus IL-12 plus IL-18) for 14 to about 16 hours, about 14 to about 15 hours, about 15 to about 16 hours, about 14 hours, about 15 hours, or about 16 hours. Various growth media can be used, such as ImmunityBio (IBRX)-NK media, which is supplemented with a cytokine cocktail.

IL-15 regulates the activation and proliferation of T and natural killer (NK) cells. IL-15 provides survival signals that maintain memory T cells in the absence of antigen. This cytokine is also implicated in NK cell development. In some embodiments, IL-15 (or a stabilized derivative thereof, for example, a superagonist such as N-803, also known or referred to as “ALT-803”) can be determined by standard methods such as stimulation of 32Dbeta cell proliferation) is present in a concentration ranging from 25-300 ng/ml, about 35 to about 290 ng/ml, about 45 to about 280 ng/ml, about 55 to about 270 ng/ml, about 65 to about 260 ng/mL, about 75 to about 250 ng/mL, about 85 to about 240 ng/ml, about 95 to about 230 ng/ml, about 105 to about 220 ng/mL, about 115 to about 210 ng/mL, about 125 to about 200 ng/ml, about 135 to about 190 ng/mL, about 145 to about 180 ng/mL, about 155 to about 170 ng/ml, about 165 ng/ml, about 25 to about 290 ng/mL, about 25 to about 280 ng/ml, about 25 to about 270 ng/ml, about 25 to about 260 ng/mL, about 25 to about 250 ng/ml, about 25 to about 240 ng/mL, about 25 to about 230 ng/ml, about 25 to about 220 ng/mL, about 25 to about 210 ng/ml, about 25 to about 210 ng/ml, about 25 to about 200 ng/mL, about 25 to about 190 ng/ml, about 25 to about 180 ng/mlm about 25 to about 170 ng/ml, about 25 to about 160 ng/ml, about 25 to about 150 ng/mL, about 25 to about 150 ng/mL, about 25 to about 140 ng/mL, about 25 to about 130 ng/mL, about 25 to about 120 ng/mL, about 25 to about 110 ng/ml, about 25 to about 100 ng/ml, about 25 to about 90 ng/ml, about 25 to about 80 ng/ml, about 25 to about 70 ng/mL, about 25 to about 60 ng/ml, about 25 to about 50 ng/mL, about 25 to about 40 ng/mL, about 30 mg/mL, about 35 to about 300 ng/ml, about 45 to about 300 ng/mL, about 55 to about 300 ng/ml, about 65 to about 300 ng/ml, about 75 to about 300 ng/ml, about 85 to about 300 ng/ml, about 95 to about 300 ng/ml, about 105 to about 300 ng/mL, about 115 to about 300 ng/ml, about 125 to about 300 ng/ml, about 135 to about 300 ng/mL, about 145 to about 300 ng/ml, about 155 to about 300 ng/ml, about 165 to about 300 ng/ml, about 175 to about 300 ng/ml, about 185 to about 300 ng/ml, about 195 to about 300 ng/ml, about 205 to about 300 ng/ml, about 215 to about 300 ng/ml, about 225 to about 300 ng/ml, about 235 to about 300 ng/mL, about 245 to about 300 ng/ml, about 255 to about 300 ng/mL, about 265 to about 300 ng/ml, about 275 to about 300 ng/mL, about 285 to about 300 ng/ml, about 295 ng/mL.

N-803 (also known or referred to as “ALT-803” or nogapendekin alfa inbakicept) is an IL-15 superagonist. N-803 is a complex consisting of human IL-15 mutant IL-15N72D (residue substitution at position 72) and IL-15Rα sushi-Fc fusion protein (see Zhu et al. J. Immunol. 2009; 183:3598-607, the relevant disclosure is hereby incorporated by reference). This results in accessory cell-independent trans-presentation of IL-15, prolonged in vivo pharmacokinetics, increased in vivo biological activity, and increased effector functions compared to IL-15. A fusion protein comprising IL-15 can also be used in the methods and composition disclosed herein as an alternative to IL-15. In one embodiment, the fusion protein is an IL-15 fused to the sushi domain of IL-15Rα. In some embodiments the IL-15 and the sushi domain of the IL-15Rα fused to the Fc domains of an antibody; see Hu et al., Discovery of a novel IL-15 based protein with improved developability and efficacy for cancer immunotherapy, Scientific Reports 8, Article number: 7675 (2018).

IL-18 helps to induce cell-mediated immunity, for example, following infection with microbial products like lipopolysaccharide (LPS). IL-18, combined with IL12, acts on CD4 T cells, CD8 T cells, and NK cells to induce IFNγ production, type II interferon that plays an important role in activating the macrophages or other cells. In embodiments, IL-18 (or an analog or stabilized analog thereof, for example, those commercially available from companies such as Miltenyi, Biolegend, In Vivogen, AkronBiotech; any recombinant cytokine can be used, for example, those produced by E. coli or CHO) can be a human recombinant IL-18. IL-18 is present in a concentration ranging from 25-50 ng/ml, e.g., 10 ng/mL, about 30 to about 45 ng/mL, about 35 to about 40 ng/mL, about 30 to about 50 ng/mL, about 40 to about 50 ng/ml, about 20 ng/mL, about 30 ng/ml, about 40 ng/ml.

IL-12 has an important role in the activity of natural killer cells and T lymphocytes. IL-12 mediates enhancement of the cytotoxic activity of NK cells and CD8+ cytotoxic T lymphocytes. IL-2 is also linked to the signal transduction of IL-12 in NK cells. Enhanced functional response of IL-12 and NK cells can be demonstrated by IFN-γ production and killing of target cells. IL-12 or analog thereof can be a human recombinant IL-12. IL-12 is present in a concentration ranging from 5-10 ng/mL, e.g., about 5 to 9 ng/mL, about 5 to 8 about ng/mL, about 5 to 7 about ng/mL, about 5 to 6 about ng/mL, about 6 to 10 about ng/mL, about 7 to about 10 ng/mL, about 8 to about 10 ng/mL, about 9 to about 10 ng/mL, about 6 to about 9 ng/mL, about 7 to about 8 ng/mL.

Thus, provided herein is a method of creating M-CENK cells from blood, the method comprising obtaining an apheresis product from a subject: isolating cells from a blood sample (e.g., mononuclear cells); contacting the isolated monocytes with one or more agents selected from the group consisting of anti-CD3 and anti-CD14, and separating the CD3+ and CD14+ cells from the cell population. The CD3−/CD14− fraction can then be expanded in growth media in the presence of a cytokine for a period of time (for example, IL-15 for 7 to 10 days). The cells can then further be cultured in growth media with a cytokine cocktail (e.g., one comprising IL-12, IL-18, and N-803) for a period of time (e.g., 14-16 hours) to produce M-CENK cells that are enriched for CD56 and CD16. These cells can then be administered to a subject or subject to cryopreservation for future administration.

### Phenotyping the M-CENK Cells

In certain embodiments, M-CENK cell populations can be assessed by detecting one or more functionally relevant markers, for example, activating receptors on NK cells. In some embodiments, one or more of CD25, CD16, CD56, DNAM-1, NKp46, NKp30, and the like can be utilized.

In some embodiments, provided herein is an M-CENK cell population comprising a population of cells that express CD56 and/or CD16 at a higher level as compared to typical M-CENK cells obtained from non-double-depleted apheresis products (i.e., created from traditional methods and not those of the present disclosure).

In embodiments, the M-CENK cell population, as described herein, can comprise about 3×, about 4×, about 5×, or more B cells compared to typical M-CENK cells obtained from non-double-depleted apheresis products. In embodiments, the M-CENK cell population described herein can comprise about 3×, about 4×, about 5×, or more NK cells compared to typical M-CENK cells obtained from non-double-depleted apheresis products. In embodiments, the M-CENK cell population described herein can comprise about 3×, about 4×, about 5×, or more dendritic cells compared to typical M-CENK cells obtained from non-double-depleted apheresis products. In embodiments, the M-CENK cell population described herein can comprise about 3×, about 4×, about 5×, or more granulocytes compared to typical M-CENK cells obtained from non-double-depleted apheresis products. In embodiments, the M-CENK cell population described herein can comprise less monocytes than typical M-CENK cells obtained from non-double-depleted apheresis products.

In some embodiments provided herein are a M-CENK cell population comprising a percentage of CD56+ cells substantially greater than M-CENK cells obtained without double-depleted apheresis methods. The M-CENK cell population comprises at least about 50%, at least about 60%, at least about 70%, at least about 80%, at least about 90%, at least about 95%, at least about 96%, at least about 97%, or higher CD56+ cells. In some embodiments, the M-CENK cell population comprises between 50-100%, 70-100%, 85-97%, 90-97%, 95-97%, or 96-97% CD56+ cells. In some embodiments, the M-CENK cell population comprises no less than 50%, no less than 70%, no less than 85%, no less than 90%, no less than 93%, or no less than 95% of the CD56+ cells.

In some embodiments, provided herein are a M-CENK cell population comprising a percentage of CD16+ cells (or cells that express CD16 to a degree) that is substantially greater than M-CENK cells obtained without double-depleted apheresis methods. The M-CENK cell population comprises at least about 50%, at least about 60%, at least about 70%, at least about 80%, at least about 90%, at least about 95%, at least about 96%, at least about 97%, at least about 98%, or about 99% of CD16+ cells. In some embodiments, the M-CENK cell population comprises between 50-100%, 70-100%, 85-100%, 90-100%, 95-100%, or 98-100% CD16+ cells. In some embodiments, the M-CENK cell population comprises no less than 50%, no less than 70%, no less than 85%, no less than 90%, no less than 93%, or no less than 95% of the CD16+ cells.

In some embodiments provided herein are a M-CENK cell population comprising a percentage of CD56+CD16+ cells (double positive) substantially greater than M-CENK cells obtained without double-depleted apheresis methods. The M-CENK cell population comprises at least about 50%, at least about 60%, at least about 70%, at least about 80%, at least about 90%, at least about 95%, at least about 96%, at least about 97%, or higher CD56+ cells. In some embodiments, the M-CENK cell population comprises between 50-100%, 70-100%, 85-97%, 90-97%, 95-97%, or 96-97% CD56+CD16+ cells. In some embodiments, the M-CENK cell population comprises no less than 50%, no less than 70%, no less than 85%, no less than 90%, no less than 93%, or no less than 95% of the CD56+CD16+ cells.

In some embodiments, provided herein are a M-CENK cell population comprising or otherwise obtained from a population of cells comprising a percentage of CD3+CD14+ cells that is substantially less than that in a typical invariant cell population obtained with methods that are not double depletion methods as described herein. The M-CENK cell population comprises at least about 50% less, at least about 60% less, at least about 70% less, at least about 80% less, at least about 90% less, at least about 95% less, at least about 96% less, at least about 97% less of CD3+CD14+ cells. In some embodiments, the M-CENK cell population comprises between 50-100%, 70-100%, 85-100%, 90-100%, 95-100%, or 98-100% of the CD3− and CD14− cells. In some embodiments, the M-CENK cell population comprises no less than 50%, no less than 70%, no less than 85%, no less than 90%, no less than 93%, or no less than 95% of the CD3− CD14− cells. In some embodiments, the M-CENK cells obtained disclosed above were cryopreserved and then thawed before use, for example, before procedures are performed to isolate exosomes from the thawed cells.

Isolation of Exosomes from the M-CENK Cells

Optionally, the exosomes of the M-CENK cells are isolated and the isolated exosomes can be administered to a patient in need thereof. Exosomes can typically be collected by ultracentrifugation from the supernatant of M-CENK cell cultures. In one illustrative example, M-CENK cell cultures are centrifuged at 300×g for 10 minutes, and the cell pellet discarded. The supernatant is centrifuged at 2000×g for 20 minutes, and the pellet (cell debris) is discarded. The resulting supernatant is subjected to ultracentrifugation at 100,000×g for 80 minutes. The resulting pellet is washed with phosphate-buffered saline and subjected to ultracentrifugation at 100,000×g for 80 minutes. The washed pellet containing exosomes can then be used directly or be formulated for administration to the patents.

To confirm the presence and the quality of the exosome preparation, techniques such as Western Blot, can be used to detect exosomeal markers, such as, Rab5B, perforin, Fas ligand (FasL), granzyme B, and granulysin. Optionally, the cytotoxicity of the exosome preparations can be analyzied by incubating with target cells, for example, Jurkat cells, for a period of time (2 hours to 20 hours). In one embodiment, the cytotoxicity of the exosomes can be determined by a Prodidium iodide (PI) assay; data can be expressed as the percent of cells that are PI positive (indicative of dead cells).

### Preparation of NK Lysates

In some embodiments, the M-CENK are lysed to produce lysates for therapeutic uses. M-CENK cells can be harvested, for example, by centrifugation, and washed with buffer (e.g., phosphate-buffered saline (PBS)). The cells are lysed in a lysis buffer, for example a Balanced Salt Solution (BSS) cell lysis buffer (BSS: 250 mM NaCl, 50 mM Tris-HCl, pH 7.4). Various methods can be used to lyse the M-CENK cells and such methods are well known in the art. In one exemplary method, the M-CENK cells are lysed by multiple cycles of freeze and thaw. In another exemplary methods, the M-CENK cells are lysed by multiple rounds of sonication. No detergent is added to prevent proteins in the lysate from denaturing. Since the perforin/granzyme pathway is Ca++ dependent, no EDTA is added. The lysate is then centrifuged to remove intact cells, insoluble cell debris and genomic DNA. The supernatant (the lysate) is collected and can be used either fresh or from cryopreserved (minus 80° C.) stock.

To confirm the quality of the lysate preparation, flow cytometric/fluorescence-activated cell sorting (FACS) cytotoxicity assays can be used to assess the effect of the cell lysate on target cells, for example, the K562 cells. Target cells can be stained using the membrane dye PKH-67GL (Sigma-Aldrich®, St. Louis, MO) and incubated with the cell lysate, and dead cells can be stained with propidium iodide. The cytotoxicity of the lysate can be determined by assessing the percent of cells that are PI positive (indicative of dead cells).

NK cells (such as aNK cells, a haNK cell, a T-haNK cells, a primary NK cell, a KHYG-1 cells) can be similarly processed to obtain NK cell lysate, aka. NK lysate.

Cytotocicity of the M-CENK Cells, the M-CENK Exosomes, and/or the M-CENK Lysates

Optionally, the cytotoxic activity of isolated or enriched M-CENK cells, the M-CENK exosomes, and/or the M-CENK lysates derived from the M-CENK cells can be assessed, e.g., in a cytotoxicity assay using tumor cells, e.g., with cultured K562, TMD-5, MS-1, OVCAR3, DAOY, THP-1, LN-18, U937, WERI-RB-1, U-118 MG, HT-29, HCC2218, KG-1, or U266 tumor cells, or the like as target cells.

Assays for evaluating cytotoxicity are well known, for example, a Calcein-AM based assay is used to measure the cytotoxic activity. of M-CENK cells towards the NK cell sensitive K562 chronic myelogenous leukemia cell line.

Calcein AM-loaded K562 (chronic myelogenous leukemia cell line) or MS-1 (a Merkel cell carcinoma cell line is resistant to resting NK cells) target cells are incubated with M-CENK effector cells at E:T ratios ranging from 20:1 to 0.02:1. Percent specific lysis of K562 target cells by M-CENK effector cells at different E:T ratio is calculated and plotted to obtain the cell killing curves.

The efficacy of the M-CENK cells, the M-CENK exosomes, and/or the M-CENK lysates on killing target cells can be evaluated with an EC₅₀, for example. EC₅₀ used in this disclosure refers to the effector to target ratio used in an assay where 50% of target cells are killed. In some embodiments, the M-CENK cells are able to kill a plurality of the target cells at an EC₅₀ of 0.1-10, e.g., 0.5-8, 1-7, 2-6, 2-5.5, or 3-7. In some embodiments, the target cell is a cell of an Acute Lymphoblastic Leukemia cell line (TMD-5), an ovarian adenocarcinoma cell line (OVCAR3), or a Merkel cell carcinoma cell line (MS-1 cell).

### Therapeutic Applications

This disclosure also provides a method to treat any type of cancer in a subject at any stage of the disease. Non-limiting examples of the suitable cancers include carcinoma, melanoma, or sarcoma. In some embodiments, the invention is used to treat cancer of hemopoietic origin such as leukemia or lymphoma. In some embodiments, the cancer is a solid tumor. In some embodiments, the invention is used to treat tumors which do not express MHC-I.

In embodiments, the cancer is a metastatic tumor. In embodiments, the tumor is a solid metastatic tumor. In embodiments, the cancer comprises a small cell lung cancer, an ovarian cancer, a breast cancer, a leukemia, a myelogenous leukemia, or a acute lymphoblastic leukemia. In embodiments, a cancer cell of a cancer according to the present disclosure is a lymphoblast. In embodiments, the cancer is a solid lung metastatic cancer, a solid liver metatastic cancer, a solid bone metastatic cancer, a solid brain metastatic cancer, or a lymph node metastatic cancer. In embodiments, the cancer is a solid thyoid metastatic cancer, a solid pancreatic metastatic cancer, s solid kidney metastatic cancer, a solid optic metastatic tumor, or a solid spleen metastatic tumor. In embodiment, the cancer can be or be derived from a melanoma, a lymphoma, a sarcoma, a genitourinary cancer, a prostate cancer, a lung cancer, a bladder cancer, a kidney cancer, a lunch cancer, a gastrointestinal tumor, or an ovarian cancer.

In some embodiments, the method to treat any type of cancer in a subject comprises administering to the patient a therapeutically effective amount of M-CENK cells as described herein, thereby treating cancer in the patient (i.e., reducing or eliminating one or more symptoms of a disease). The M-CENK cells are created from a population of immune cells isolated by methods as described herein, wherein the M-CENK cells comprise a greater amount of CD16 expression than M-CENK cells created by traditional apheresis methods and are CD56 enriched.

The disclosure also provides a method to treat cancer, the method comprising administering to the patient a therapeutically effective amount of M-CENK cells, the M-CENK exosomes, and/or the M-CENK lysates as described herein, thereby treating cancer. The M-CENK cells are created from a population of immune cells resulting from a CD3−/CD14− double depletion apheresis methodology, wherein the M-CENK cells exhibit greater CD16 expression and are CD56-enriched compared to M-CENK cells created from products of traditional apheresis methodology.

Also provided are methods of treating a subject in need thereof with M-CENK cells as described herein. In some embodiments, the subject or patient suffers from cancer or an infectious disease, such as a viral infection.

The M-CENK cells can be administered to an individual (i.e., a subject, especially a mammalian subject having or suspected of having a cancer) by absolute numbers of cells. e.g., 0.25×10⁸, 0.50×10⁸, 0.75×10⁸, 0.1×10⁹, 0.25×10⁹, 0.50×10⁹, 0.60×10⁹, 0.65×10⁹, 0.70×10⁹, 0.75×10⁹, 0.8×10⁹, 0.9×10⁹, 1×10⁹, 1.25×10⁹, 1.50×10⁹, 2.0×10⁹, 2.25×10⁹, 5.0×10⁹, 6.0×10⁹, 7.0×10⁹, 8.0×10⁹, 9.0×10⁹, or 1.0×10¹⁰, or a number within a range defined by any two of the numbers above, endpoints inclusive. In some embodiments, said individual can be administered from about 250 million cells/injection (i.e., 0.25×10⁹) to up to about 750 million cells/injection (0.75×10⁹). Therefore, this disclosure also provides a composition comprising a plurality of M-CENK cells, wherein the number of cells is 0.25×10⁸, 0.50×10⁸, 0.75×10⁸, 0.1×10⁹, 0.25×10⁹, 0.50×10⁹, 0.60×10⁹, 0.65×10⁹, 0.70×10⁹, 0.75×10⁹, 0.8×10⁹, 0.9×10⁹, 1×10⁹, 1.25×10⁹, 1.50×10⁹, 2.0×10⁹, 2.25×10⁹, 5.0×10⁹, 6.0×10⁹, 7.0×10⁹, 8.0×10⁹, 9.0×10⁹, or 1.0×10¹⁰, or a number within a range defined by any two of the numbers above, endpoints inclusive. In some embodiments, the number of the plurality of M-CENK cells in the composition is from about 250 million cells/injection (i.e., 0.25×10⁹) to up to about 750 million cells/injection (0.75×10⁹).

In other embodiments, said individual can be administered from about 250 million cells/injection/m² (i.e., 0.15e9 cells/injection/m²) to up to about 790 million cells/injection/m² (i.e., 0.795e9 cells/injection/m²), such as at about, at least about, or at most about, 0.15e9/m², 0.25e9/m², 0.30e9/m², 0.35e9/m², 0.40e9/m², 0.45e9/m², 0.50e9/m², 0.55e9/m², 0.60e9/m², 0.65e9/m², 0.70e9/m², 0.75e9/m², or 0.79e9/m^{2 2} (and so forth) M-CENK cells per injection, or a dosage within a range defined by any two of the numbers above, endpoints inclusive.

In other embodiments, M-CENK cells can be administered to such individual by relative numbers of cells, e.g., said individual can be administered about 150 million cells (i.e., 0.15e9 cells) to up to about 790 million cells (i.e., 0.79e9 cells) per kilogram of the individual, such as at about, at least about, or at most about, 0.15e9, 0.25e9, 0.30e9, 0.35e9, 0.40e9, 0.45e9, 0.50e9, 0.55e9, 0.60e9, 0.65e9, 0.70e9, 0.75e9, or 0.79e9 (and so forth) M-CENK cells per kilogram of the individual, or or a number within a range defined by any two of the numbers above, endpoints inclusive.

In other embodiments, the total dose may be calculated by m² of body surface area, including about 0.15e9, 0.25e9, 0.30e9, 0.35e9, 0.40e9, 0.45e9, 0.50e9, 0.55e9, 0.60e9, 0.65e9, 0.70e9, 0.75e9, or 0.79e9, per m², or any ranges between any two of the numbers, endpoints inclusive. The average person (i.e., a human subject) is about 1.6 to about 1.8 m². In a preferred embodiment, between about 1 billion and about 3 billion M-CENK cells are administered to a patient. In other embodiments, the amount of M-CENK cells injected per dose may calculated by m² of body surface area, including 0.15e9, 0.25e9, 0.30e9, 0.35e9, 0.40e9, 0.45e9, 0.50e9, 0.55e9, 0.60e9, 0.65e9, 0.70e9, 0.75e9, or 0.79e9, or a number within a range defined by any two of the numbers above, endpoints inclusive, cells per m². The average body surface area for a person is 1.6-1.8 m².

In other embodiments, M-CENK cells can be administered to such individual by relative numbers of cells, e.g., said individual can be administered about 250 million cells (i.e., 0.15e9 cells) to up to about 790 million cells (i.e., 0.79e9 cells) per kilogram of the individual, such as at about, at least about, or at most about, 0.15e9, 0.25e9, 0.30e9, 0.35e9, 0.40e9, 0.45e9, 0.50e9, 0.55e9, 0.60e9, 0.65e9, 0.70e9, 0.75e9, or 0.79e9 (and so forth) M-CENK cells per kilogram of the individual, or any ranges between any two of the numbers, endpoints inclusive.

An effective number of exosomes can be administered on the basis of body surface area to be covered (e.g., affected area), for example in a topical composition. A suitable dose range is from about 0.001 mg to about 100 mg of equivalent per m2 body surface area of a tumoricidal and/or antimicrobial component, for instance from about 0.005 mg/m2 to about 50 mg/m². The dosage can be administered daily, such as once, twice, three times or more per day, or every two or several days, or every week, etc. The frequency of administration can be reduced if an extended-release formulation is administered.

In one embodiment, the effective amount can be administered on the basis of tumor volume. A suitable dose range is from about 1:100 to about 1:10,000 exosome/microvesicle preparation to tumor volume ratio. In one embodiment, the suitable dose range from about 1:100 to about 1:1,000 exosome/microvesicle preparation to tumor volume ratio. In one embodiment, the suitable doses range from about 1:1,000 to about 1:10,000 exosome/microvesicle preparation to tumor volume ratio.

In one embodiment, the effective amount can be administered based on patient body weight. A suitable dose range is from about 1 μg to about 100 mg exosome preparation per kg body weight. In one embodiment, the suitable dose range from about 1 μg to about 10 mg exosome preparation per kg body weight. In one embodiment, the suitable dose range from about 1 μg to about 100 μg exosome preparation per kg body weight. In one embodiment, the suitable dose range from about 10 μg to about 10 mg exosome/microvesicle preparation per kg body weight. In one embodiment, the suitable doses range from about 100 μg to about 10 mg exosome/microvesicle preparation per kg body weight.

M-CENK cells, the M-CENK exosomes, and/or the M-CENK lysates can be administered once to a patient with cancer or they can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, endpoints inclusive.

In some embodiments, M-CENK cells, the M-CENK exosomes, and/or the M-CENK lysates are administered in a composition comprising the M-CENK cells and a medium (such as human albumin or an equivalent) thereof. In some embodiments, the medium comprises human albumin. In some embodiments, the medium comprises about 1% to about 15% human albumin or human albumin equivalent. In some embodiments, the medium comprises about 1% to 10% human albumin or human albumin equivalent. In some embodiments, the medium comprises about 1% to about 5% human albumin or human albumin equivalent. In one embodiment, the medium comprises about 2.5% human albumin or human albumin equivalent. In some embodiments, the albumin is human plasma. In some embodiments, a recombinant albuminsubstitute that is acceptable for use in human therapeutics is used instead of human albumin. Such albumin substitutes may be known in the art or developed in the future. Although concentrations of human albumin over 15% can be used, it is contemplated that concentrations greater than about 5% will be cost-prohibitive. In some embodiments, M-CENK cells, the M-CENK exosomes, and/or the M-CENK lysates are administered in a composition comprising M-CENK cells, the M-CENK exosomes, and/or the M-CENK lysates and an isotonic liquid solution that supports cell viability. In some embodiments, M-CENK cells are administered in a composition reconstituted from a cryopreserved sample.

Pharmaceutically acceptable compositions comprising the M-CENK cells, the M-CENK exosomes, and/or the M-CENK lysates can include a variety of pharmaceutically acceptable carriers and excipients. Various aqueous carriers can be used, e.g., buffered saline and the like. These solutions are sterile and generally free of undesirable matter. Suitable carriers and excipients and their formulations are described in Remington: The Science and Practice of Pharmacy, 21st Edition, David B. Troy, ed., Lippicott Williams & Wilkins (2005). A pharmaceutically acceptable carrier means a material that is not biologically or otherwise undesirable, i.e., the material is administered to a subject without causing undesirable biological effects or interacting deleteriously with the other components of the pharmaceutical composition in which it is contained. If administered to a subject, the carrier is optionally selected to minimize degradation of the active ingredient and to minimize adverse side effects in the subject. As used herein, the term pharmaceutically acceptable is used synonymously with physiologically acceptable and pharmacologically acceptable. A pharmaceutical composition will generally comprise agents for buffering and preservation in storage and can include buffers and carriers for appropriate delivery, depending on the route of administration.

These compositions for use in in vivo or in vitro may be sterilized by sterilization techniques employed for cells. The compositions may contain acceptable auxiliary substances required to approximate physiological conditions such as pH adjusting and buffering agents, toxicity adjusting agents, and the like, for example, sodium acetate, sodium chloride, potassium chloride, calcium chloride, sodium lactate, and the like. The concentration of cells in these formulations and/or other agents can vary. It will be selected primarily based on fluid volumes, viscosities, body weight, and the like in accordance with the particular mode of administration selected and the subject's needs.

In one embodiment, M-CENK cells, the M-CENK exosomes, and/or the M-CENK lysates are administered to the patient in conjunction with one or more other treatments or agent for the cancer being treated. In some embodiments, the one or more other treatments for the cancer being treated include, for example, an antibody, radiation, chemotherapeutic, stem cell transplantation, or hormone therapy.

In some embodiments, M-CENK cells, the M-CENK exosomes, and/or the M-CENK lysates and the other cancer agent/treatment are administered simultaneously or approximately simultaneously (e.g., within about 1, 5, 10, 15, 20, or 30 minutes of each other). In some embodiments, the M-CENK cells and the other cancer agent/treatment are administered sequentially. In some embodiments, the other cancer treatment/agent is administered one, two, or three days after the administration of the M-CENK cells. In embodiments, co-therapies can be or otherwise comprise co-treatment with an IL-15, a fusion protein consisting of IL-15 fused to the sushi domain of IL-15Rα, or N-803. In some embodiments, the M-CENK cells is co-administered with an expression construct expressing one of the IL-15, a fusion protein comprising IL-15 fused to the sushi domain of IL-15Rα, a fusion protein comprising a sushi domain of the IL-15Rα fused to the Fc domains of an antibody, or N-803. In some embodiments, the other agent is a histone deacetylase (HDAC) inhibitor. In some embodiments, the other agent is a class 1 HDAC. In some embodiments, the other agent is a fusion protein comprising at least one of a Transforming Growth Factor beta (TGF-β) trap (TGFβRII), a tumor necrosis targeted (TNT) antibody, IL-12, an IgG domain, and LAIR-II.

In one embodiment, the other cancer agent is an antibody. In one embodiment, M-CENK cells, the M-CENK exosomes, and/or the M-CENK lysates are administered in conjunction with an antibody targeting the diseased cells. In one embodiment, M-CENK cells, the M-CENK exosomes, and/or the M-CENK lysates are administered in conjunction with an antibody targeting a checkpoint inhibitor. In one embodiment, M-CENK cells, the M-CENK exosomes, and/or the M-CENK lysates and an antibody are administered to the patient together, e.g., in the same formulation; separately, e.g., in separate formulations, concurrently; or can be administered separately, e.g., on different dosing schedules or at different times of the day. When administered separately, the antibody can be administered via any suitable route, such as intravenous or intra-tumoral injection.

In some embodiments, M-CENK cells, the M-CENK exosomes, and/or the M-CENK lysates of the present disclosure are used in combination with therapeutic antibodies and/or other anti-cancer agents. Therapeutic antibodies may be used to target cells that express cancer-associated or tumor-associated markers. Examples of cancer therapeutic monoclonal antibodies are shown in Table 1.

TABLE 1
Illustrative therapeutic monoclonal antibodies
Examples of FDA-approved therapeutic monoclonal antibodies

| | Brand | | | Indication |
|---|---|---|---|---|
| Antibody | name | Company | Target | (Targeted disease) |
| Alemtuzumab | Campath ® | Genzyme | CD52 | Chronic lymphocytic |
| | | | | leukemia |
| Brentuximab | Adcetris ® | | CD30 | Anaplastic large cell |
| vedotin | | | | lymphoma (ALCL) |
| | | | | and Hodgkin lymphoma |
| Cetuximab | Erbitux ® | Bristol-Myers | epidermal growth | Colorectal cancer, Head and |
| | | Squibb/Eli | factor receptor | neck cancer |
| | | Lilly/Merck | | |
| | | KGaA | | |
| Gemtuzumab | Mylotarg ® | Wyeth | CD33 | Acute myelogenous |
| | | | | leukemia (with calicheamicin) |
| Ibritumomab | Zevalin ® | Spectrum | CD20 | Non-Hodgkin |
| tiuxetan | | Pharmaceuticals, | | lymphoma (with yttrium- |
| | | Inc. | | 90 or indium-111) |
| Ipilimumab | Yervoy ® | | blocks CTLA-4 | Melanoma |
| (MDX-101) | | | | |
| Ofatumumab | Arzerra ® | | CD20 | Chronic lymphocytic |
| | | | | leukemia |
| Palivizumab | Synagis ® | MedImmune | an epitope of the | Respiratory Syncytial Virus |
| | | | RSV F protein | |
| Panitumumab | Vectibix ® | Amgen | epidermal growth | Colorectal cancer |
| | | | factor receptor | |
| Rituximab | Rituxan ®, | Biogen | CD20 | Non-Hodgkin lymphoma |
| | Mabthera ® | Idec/Genentech | | |
| Tositumomab | Bexxar ® | GlaxoSmithKline | CD20 | Non-Hodgkin lymphoma |
| Trastuzumab | Herceptin ® | Genentech | ErbB2 | Breast cancer |
| Blinatunomab | | | bispecific CD19− | Philadelphia chromosome- |
| | | | directed CD3 T-cell | negative relapsed or |
| | | | engager | refractory B cell precursor |
| | | | | acute lymphoblastic |
| | | | | leukemia (ALL) |
| Avelumamab | | | anti-PD-L1 | Non-small cell lung cancer, |
| | | | | metastatic Merkel cell |
| | | | | carcinoma; gastic cancer, |
| | | | | breast cancer, ovarian |
| | | | | cancer, bladder cancer, |
| | | | | melanoma, meothelioma, |
| | | | | including metastatic or |
| | | | | locally advanced solid |
| | | | | tumors |
| Daratumumab | | | CD38 | Multiple myeloma |
| Elotuzumab | | | a SLAMF7- | Multiple myeloma |
| | | | directed (also | |
| | | | known as CD 319) | |
| | | | immunostimulatory | |
| | | | antibody | |

In some embodiments, M-CENK cells, the M-CENK exosomes, and/or the M-CENK lysates of the present disclosure are used in combination with the antibodies, such as the IL-8 antibodies, as described in U.S. Pat. Nos. 10,752,682 and 11,773,158.

Administration of such M-CENK cells, the M-CENK exosomes, and/or the M-CENK lysates may be carried out simultaneously with the administration of the monoclonal antibody, or in a sequential manner. In some embodiments, the M-CENK cells are administered to the subject after the subject has been treated with the monoclonal antibody. Alternatively, the M-CENK cells may be administered at the same time, e.g., within 24 hours, of the monoclonal antibody.

In some embodiments, M-CENK cells, the M-CENK exosomes, and/or the M-CENK lysates are administered intravenously. In some embodiments the M-CENK cells, the M-CENK exosomes, and/or the M-CENK lysates are infused directly into the bone marrow.

Therefore, this disclosure provides a method of treating cancer or viral infection in a patient in need thereof, the method comprising administering to the patient a therapeutically effective amount of M-CENK cells, the M-CENK exosomes, and/or the M-CENK lysates from the population of M-CENK cells using the methods disclosed herein to thereby treat cancer.

In some embodiments, M-CENK cells, the M-CENK exosomes, and/or the M-CENK lysates are administered to the patient in conjunction with one or more viral vectors, each expressing one or more immunogenic peptides and/or peptides having therapeutic effects. In some embodiments, at least one of the viral vectors is an adenoviral vector. In some embodiments, at least one of the viral vectors is a replication defective adenovirus vector, which comprises a deletion in the E2b region.

In some embodiments, M-CENK cells, the M-CENK exosomes, and/or the M-CENK lysates are administered to the patient in conjunction NK cell lysate, such as those prepared from aNK cells, haNK cells, T-haNK cells, primary NK cells, KHYG-1 cells, etc.

### Kits

Also disclosed are kits for the treatment of cancer or an infectious disease using compositions comprising an amount of M-CENK cells, the M-CENK exosomes, and/or the M-CENK lysates as described herein. In some embodiments, the kits of the present disclosure may also include at least one monoclonal antibody. In embodiments, the amount of M-CENK cells, the M-CENK exosomes, and/or the M-CENK lysatesis an amount to reduce or eliminate one or more symptoms of a disease, such as cancer. M-CENK cells, the M-CENK exosomes, and/or the M-CENK lysates can further be provided for in unit dose forms in kits according to the present disclosure. In some embodiments, a kit of the present disclosure comprises an IL-15 or a fusion protein comprising IL-15, for example, a fusion protein comprising an IL-15 bound (covalently or non-covalently) to the sushi domain of IL-15Rα, or a fusion protein comprising IL-15 and the sushi domain of the IL-15Rα fused to the Fc domains, or N-803. In some embodiments, the kit comprises one or more of checkpoint inhibitor antibodies or tumor targeted antibodies, for example, those described in U.S. Pat. No. 11,384,157.

In certain embodiments, the kit may contain additional compounds such as therapeutically active compounds or drugs that are to be administered before, at the same time or after administration of M-CENK cells. Examples of such compounds include an antibody, vitamins, minerals, fludrocortisone, ibuprofen, lidocaine, quinidine, chemotherapeutic, etc.

In various embodiments, instructions for use of the kits will include directions to use the kit components in the treatment of a cancer or an infectious disease. The instructions may further contain information regarding how to handle M-CENK cells (e.g., thawing and/or culturing). The instructions may further include guidance regarding the dosage and frequency of administration.

In certain embodiments, the kit further comprises one or more containers filled with one or more compositions described herein, e.g., a composition comprising M-CENK cells, the M-CENK exosomes, and/or the M-CENK lysates as described herein. Optionally associated with such containers can be a label indicating the kit is for treating a cancer, such as those described herein. Optionally the label also includes a notice in the form prescribed by a governmental agency regulating the manufacture, use or sale of pharmaceuticals or biological products, which notice reflects approval by the agency of manufacture, use or sale for human administration.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed while specific reference of each various individual and collective combinations and permutations of these compounds may not be explicitly disclosed, each is specifically contemplated and described herein. For example, if a method is disclosed and discussed and several modifications that can be made to several molecules, including the method, are discussed, each and every combination and permutation of the method and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods and that each such combination or subset of combinations is specifically contemplated and should be considered disclosed.

### EMBODIMENTS

This disclosure contains the following exemplary, nonlimiting embodiments:
Embodiment 1. A method of generating memory-like cytokine enhanced natural killer (M-CENK) cells from a blood sample, the method comprising: (a) removing CD3+ cells and CD14+ cells from the blood sample to obtain a CD3− CD14− population of cells; (b) incubating the CD3− CD14− population of cells with a first cytokine composition comprising a stabilized IL-15 analog or fusion protein comprised thereof for a first period (optionally 8-10 days), and (c) incubating the CD3− CD14− population of cells with a second cytokine composition comprising i) IL-15 or a stabilized derivative thereof, ii) IL-18 or a stabilized derivative thereof, and iii) IL-12 or a stabilized derivative thereof for a second period (optionally 14-16 hours), thereby producing a population of CD56+ enriched m-ceNK cells.
Embodiment 2. The method of embodiment 1, wherein the IL-15 or stabilized derivative thereof is in a concentration ranging from 25 to 175 ng/mL.
Embodiment 3. The method of embodiment 1, wherein the IL-18 or stabilized derivative thereof is in a concentration ranging from 25 to 50 ng/mL.
Embodiment 4. The method of embodiment 1, wherein the IL-12 or stabilized derivative thereof is in a concentration ranging from 5 to 10 ng/mL.
Embodiment 5. The method of embodiment 1, wherein the blood sample is obtained from a cancer patient.
Embodiment 6. The method of embodiment 5, wherein a cancer of the cancer patient is a small cell lung cancer, an ovarian cancer, a breast cancer, a leukemia, a myelogenous leukemia, or an acute lymphoblastic leukemia.
Embodiment 7. The method of embodiment 1, wherein step (a) is by magnetic cell separation.
Embodiment 8. The method of embodiment 1, wherein the first period lasts 7 to 12 days.
Embodiment 9. The population of CD56+ enriched cells produced from the method of any one of embodiments 1-8.
Embodiment 10. The population of CD56+ enriched cells of embodiment 9, wherein greater than 90% of the cells are CD56+.
Embodiment 11. The population of CD56+ enriched cells of embodiment 9, wherein greater than 60% of the cells are CD16+.
Embodiment 12. A method of treating a patient diagnosed with cancer, the method comprising: (a) obtaining a blood sample from the patient; (b) removing CD3+ cells and CD14+ cells from the blood sample to obtain a CD3− CD14− fraction; (c) contacting the CD3− CD14− fraction of cells with IL-15 or a stabilized derivative thereof for a first period of time; (d) contacting the CD3− CD14− fraction of cells with IL-12 or a derivative thereof, IL-18 or a derivative thereof, and IL-15 or a derivative thereof, for a second period of time, thereby obtaining a population of CD56+ enriched M-CENKcells; (e) contrifuging the cell culture to produce a supernant, (f) isolating exosomes from the supernatant; and (g) administering the isolated exosomes to the patient, thereby treating the cancer.
Embodiment 13. A method of treating a patient diagnosed with cancer, the method comprising administering a therapeutically effective amount of the CD56+ enriched M-CENK cells of embodiment 9 to the patient.
Embodiment 14. The method of embodiment 12, The method of embodiment 12, wherein the cancer is a solid tumor, optionally the cancer is a metastatic solid tumor.
Embodiment 15. The method of embodiment 12, wherein the cancer is a small cell lung cancer, an ovarian cancer, a breast cancer, a leukemia, a myelogenous leukemia, or a acute lymphoblastic leukemia.
Embodiment 16. The method of any one of embodiments 12-15, wherein greater than 90% of the CD56+ enriched population of cells are CD56+.
Embodiment 17. The method of any one of embodiments 12-16, wherein greater than 60% of the CD56+ enriched population of cells are CD16+.
Embodiment 18. The method of embodiment 12-17, wherein the method further comprises administering an antibody.
Embodiment 19. The method of embodiment 18, wherein the antibody is an anti-CD20 antibody.
Embodiment 20. A kit for preparing CD56+M-CENK cell exosomes, the kit comprising: i) reagents for depleting CD3+ cells and CD14+ cells from a blood sample; ii) IL-15 or a fusion protein comprised thereof, iii) IL-12 or a fusion protein comprised thereof, and iv) IL-18 or a fusion protein comprised thereof.
Embodiment 21. A population of CD56+ enriched M-CENK cells wherein at least 90% of the cells are CD3−, CD14−, and CD56+, and wherein at least 60% of the cells express CD16.
Embodiment 22. The population of CD56+ enriched M-CENK cells of embodiment 21, wherein the cells are freezed and thawed.
Embodiment 23. A cell culture mixture comprising a population of blood cells that are CD3− CD14−, a first cytokine composition comprising a stabilized IL-15 analog or fusion protein.
Embodiment 24. The cell culture mixture of embodiment 25, further comprising a second cytokine composition comprising one or more of i) IL-15 or a stabilized derivative thereof, ii) IL-18 or a stabilized derivative thereof, and iii) IL-12 or a stabilized derivative.
Embodiment 25 Exosomes isolated from CD56+ enriched M-CENK cells for use in the treatment of cancer, wherein said exosomes are obtained by: (a) obtaining a blood sample from a cancer patient; (b) removing CD3+ cells and CD14+ cells to obtain a CD3− CD14− fraction; (c) contacting the CD3− CD14− fraction with IL-15 or a stabilized derivative thereof for a first period of time (optionally 8-10 days); (d) contacting the CD3− CD14− fraction with IL-12 or a derivative thereof, IL-18 or a derivative thereof, and IL-15 or a derivative thereof for a second period of time (optionally 14-16 hours) to obtain a cell culture comprising a population of CD56+ enriched M-CENK cells; and (e) isolating exosomes from the cell culture.
Embodiment 26. Use of exosomes isolated from CD56+ enriched M-CENK cells for the manufacture of a medicament for the treatment of cancer, obtainable by a process comprising: (a) obtaining a blood sample from a cancer patient; (b) removing CD3+ cells and CD14+ cells to obtain a CD3− CD14− fraction; (c) contacting the CD3− CD14− fraction with IL-15 or a stabilized derivative thereof for a first period of time (optionally 8-10 days); (d) contacting the CD3− CD14− fraction with IL-12 or a derivative thereof, IL-18 or a derivative thereof, and IL-15 or a derivative thereof for a second period of time (optionally 14-16 hours) to obtain a cell culture comprising a population of CD56+ enriched M-CENK cells; and (e) isolating exosomes from the cell culture.
Embodiment 27. Use of exosomes isolated from CD56+ enriched M-CENK cells or use of lysates derived for the treatment of cancer, obtainable by a process comprising: (a) obtaining a blood sample from a cancer patient; (b) removing CD3+ cells and CD14+ cells to obtain a CD3− CD14− fraction; (c) contacting the CD3− CD14− fraction with IL-15 or a stabilized derivative thereof for a first period of time (optionally 8-10 days); (d) contacting the CD3− CD14− fraction with IL-12 or a derivative thereof, IL-18 or a derivative thereof, and IL-15 or a derivative thereof for a second period of time (optionally 14-16 hours) to obtain obtain a cell culture comprising a population of CD56+ enriched M-CENK cells; and (e) isolating exosomes from the cell culture or preparing lysates from the population of CD56+ enriched M-CENK cells.
Embodiment 28. A pharmaceutical composition comprising i) exosomes isolated from CD56+ enriched M-CENK cells or lysates derived from CD56+ enriched M-CENK cells and ii) a pharmaceutically acceptable carrier, for use in treatment of cancer, wherein the exosomes are obtained by a process comprising: (a) obtaining a blood sample from a cancer patient; (b) removing CD3+ cells and CD14+ cells to obtain a CD3− CD14− fraction; (c) contacting the CD3− CD14− fraction with IL-15 or a stabilized derivative thereof for a first period of time (optionally 8-10 days); (d) contacting the CD3− CD14− fraction with IL-12 or a derivative thereof, IL-18 or a derivative thereof, and IL-15 or a derivative thereof for a second period of time (optionally 14-16 hours) to obtain a cell culture comprising a population of CD56+ enriched M-CENK cells; and (e) isolating exosomes from the cell culture or preparing lysates from the CD56+ enriched M-CENK cells.
Embodiment 29. A kit comprising exosomes isolated from CD56+ enriched M-CENK cells, and instructions for use in the treatment of cancer, wherein the exosomes are obtained by a process comprising: (a) obtaining a blood sample from a cancer patient; (b) removing CD3+ cells and CD14+ cells to obtain a CD3− CD14− fraction;
(c) contacting the CD3− CD14− fraction with IL-15 or a stabilized derivative thereof for a first period of time (optionally 8-10 days); (d) contacting the CD3− CD14− fraction with IL-12 or a derivative thereof, IL-18 or a derivative thereof, and IL-15 or a derivative thereof for a second period of time (optionally 14-16 hours) to obtain obtain a cell culture comprising a population of CD56+ enriched M-CENK cells; and (e) isolating exosomes from the cell culture.
Embodiment 30. A kit for preparing CD56+M-CENK cell lysates, the kit comprising: i) reagents for depleting CD3+ cells and CD14+ cells from a blood sample; ii) IL-15 or a fusion protein comprised thereof, iii) IL-12 or a fusion protein comprised thereof, and iv) IL-18 or a fusion protein comprised thereof, and v) a lysis buffer.
Embodiment 31. Use of lysates prepared from CD56+ enriched M-CENK cells for the manufacture of a medicament for the treatment of cancer, obtainable by a process comprising: (a) obtaining a blood sample from a cancer patient; (b) removing CD3+ cells and CD14+ cells to obtain a CD3− CD14− fraction; (c) contacting the CD3− CD14− fraction with IL-15 or a stabilized derivative thereof for a first period of time (optionally 8-10 days); (d) contacting the CD3− CD14− fraction with IL-12 or a derivative thereof, IL-18 or a derivative thereof, and IL-15 or a derivative thereof for a second period of time (optionally 14-16 hours) to obtain a population of CD56+ enriched M-CENK cells; and (e) preparing lysates from the M-CENK cells.
Embodiment 27. Use of lysates prepared from CD56+ enriched M-CENK cells for the treatment of cancer, obtainable by a process comprising: (a) obtaining a blood sample from a cancer patient; (b) removing CD3+ cells and CD14+ cells to obtain a CD3− CD14− fraction; (c) contacting the CD3− CD14− fraction with IL-15 or a stabilized derivative thereof for a first period of time (optionally 8-10 days); (d) contacting the CD3− CD14− fraction with IL-12 or a derivative thereof, IL-18 or a derivative thereof, and IL-15 or a derivative thereof for a second period of time (optionally 14-16 hours) to obtain a population of CD56+ enriched M-CENK cells; and (e) preparing lysates from the M-CENK cells.
Embodiment 28. A pharmaceutical composition comprising lysates prepared from CD56+ enriched M-CENK cells and a pharmaceutically acceptable carrier, for use in the treatment of cancer, wherein the exosomes are obtained by a process comprising: (a) obtaining a blood sample from a cancer patient; (b) removing CD3+ cells and CD14+ cells to obtain a CD3− CD14− fraction; (c) contacting the CD3− CD14− fraction with IL-15 or a stabilized derivative thereof for a first period of time (optionally 8-10 days); (d) contacting the CD3− CD14− fraction with IL-12 or a derivative thereof, IL-18 or a derivative thereof, and IL-15 or a derivative thereof for a second period of time (optionally 14-16 hours) to obtain a population of CD56+ enriched M-CENK cells; and (e) preparing lysates from the M-CENK cells.

### EXAMPLES

The following examples are for illustrative purposes only and should not be interpreted as limitations. There are a variety of alternative techniques and procedures available to those of skill in the art that would similarly permit one to perform the examples below successfully.

### Example 1: Overview of Processes According to the Present Disclosure

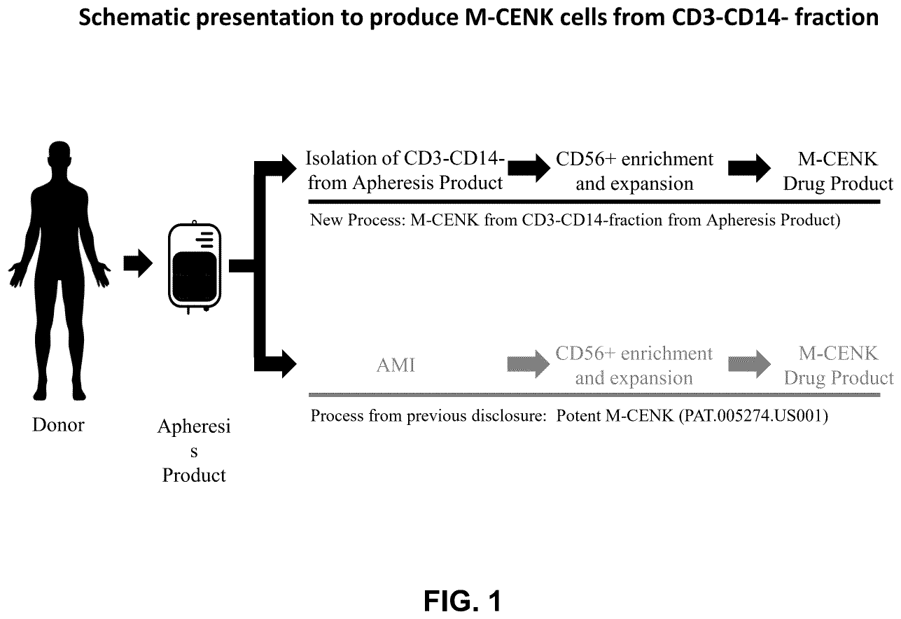

FIG. 1 is a schematic representation of previously described method (see, US 2024/0132844) of producing M-CENK cells from apheresis material (bottom flow chart) and an embodiment of a method of producing M-CENK cells from a CD3−/CD14− double-depleted apheresis product (top flow chart). According to the processes in FIG. 1, apheresis material was collected from a healthy donor and sent to an internal cell processing laboratory for transferring the apheresis material intoApheresis Material Intermediate (AMI) bags (6×40 mL cryobags). Some notable parameters that were observed between the processes described herein (top flow portion of FIG. 1 compared to AMI processing methods shown in the bottom portion of FIG. 1) are as follows (Table 2):

TABLE 2
Notable Parameter Differences Between Double Depletion Process and Traditional Process:

| | | | Parameters | | | |
|---|---|---|---|---|---|---|
| | | | | | % CD16 | |
| | | Starting | | | Expression on CD56+ | |
| | | Cell | NK | NK | cells at harvest | |
| Subject | Starting Material | Population | Expansion | Enrichment | CENK | M-CENK |
| Healthy | Fresh Apheresis | CD3− | ~1.5 × 10⁶ | Faster | 78.70 | 73.00 |
| Donor | Product | CD14− | cells/mL in | ~95% CD56+ | | |
| | (New Process | Cells | 8 days | Enrichment | | |
| | as provided in | | | in 8 days | | |
| | this application) | | | | | |
| | Cryopreserved | MNC | ~7.7 × 10⁵ | ~58% CD56+ | 62.80 | 38.00 |
| | Apheresis Material | | cells/mL in | Enrichment | | |
| | Intermediate (AMI) | | 8 days | in 14 days | | |
| | (Existing process | | | | | |
| | as disclosed in | | | | | |
| | US2024/013284 | | | | | |

### Example 2: Use of CD3−/CD14− Double-depleted Apheresis Material for Generation of Unique M-CENK Cells

Apheresis product derived from a newly diagnosed cancer patient is depleted of CD3+ and CD14+ cells using a magnetic cell separation platform (CliniMACS® Plus platform). Cells are expanded in culture vessels in the presence of IL-15 or N-803 for 7-12 days to create CENK cells and then stimulated with a cytokine cocktail comprising N-803 (ImmunityBio, 175 ng/mL), IL-18 (Invivogen, 50 ng/ml), or IL-12 (BioLegend, 10 ng/ml) for 14-16 hours to generate M-CENK cells. The G-Rex-Xuri bioreactors were used as the culture vessels in these experiments. Cells are ready for therapeutic administration. This is compared to the use of a thawed apheresis product, which is expanded in a G-Rex for 14 days. There was no notable difference in results using conventional methods between frozen and fresh cells (data not shown). A population of NK (CD56+) cells is produced in both conditions. However, the CD3/CD14 depleted cells show higher CD16 expression, increased cell killing, and antibody-dependent cellular toxicity (ADCC).

### Materials and Methods

According to standard apheresis collection procedures, an apheresis product was collected from a donor as a continuous mononuclear cell (MNC) collection. Collected MNC cells were centrifuged in a cell bag at 200×g at room temperature for 15 minutes. Supernatant was removed from the cell bag using a plasma extractor (Vendor: Fenwal, Model: 4R4414). And the cell pellet was resuspended using buffer comprised of PBS/EDTA containing 1.0% Human Albumin.

To prevent non-specific antibody binding, 1% IVIG was aseptically added to the remaining cell solution and incubated at room temperature for 6 minutes. After incubation, cells were stained with an anti-CD3 reagent (Anti-CD3 agent is a CliniMACS® CD3 GMP Microbeads from Miltenyi and used at 7% v/v of the cell solutions). The cells were incubated at room temperature for 30 minutes on a plate shaker at 50 rpm. After incubation with the anti-CD3 reagent, the cells were washed by centrifugation at 300×g at brake setting 3 for 15 minutes at room temperature. A CliniMACS LS Tubing set contain attachment was used for collection. After centrifugation, the supernatant was removed using a plasma extractor.

The residual Cell pellet was re-suspended in PBS/EDTA/0.5% HA buffer and passed through an In-Line Filter Adapter (200 μm, Miltenyi) into a new transfer pack to remove cell aggregates, if any. A cell bag containing cells stained with an anti-CD3 antibody was aseptically attached to the CliniMACS® LS Tubing Set. CliniMACS® Plus was operated based on the Depletion 2.1 program. The CD3 depleted fraction was then collected in a cell bag and the cell bag containing the CD3 depleted fraction was then aseptically removed, centrifuged at room temperature at 300×g, and resuspended in PBS/EDTA/0.5% HA buffer. After centrifugation, the supernatant was removed using a plasma extractor.

Washed cells were stained with a CD14 reagent (CD14 reagent is a CliniMACS® CD14 GMP Microbeads from Miltenyi) at room temperature for 30 minutes on a plate shaker at 50 rpm. Stained cells were washed with same buffer (PBS/EDTA/0.5% HA buffer) by centrifugation at 300×g at room temperature for 15 minutes. After centrifugation, the supernatant was removed using a plasma extractor.

Inside the biological safety cabinet (BSC), the cell bag was aseptically assembled with CliniMACS® LS Tubing Set CliniMACS® Plus was operated using ENRICHMENT 1.1 program. At the end of the CD14⁺ selection process, non-selected cells (CD14⁻) were collected in a bag. This CD3/CD14 double-depleted fraction was then washed and suspended in IB NK growth media (i.e., NK-XM growth media from Immunity Bio, comprising CTS NK-Xpander media containing 10% Human AB serum plus CTS NK-Xpander supplements and N-803) and used for CD56⁺ cell enrichment and expansion. CD56⁺ cells were enriched and expanded IB media in G-Rex flasks. The cell phenotype was monitored throughout the process by flow-cytometry based phenotyping assay.

Following enrichment, the cells were stimulated with a cytokine cocktail containing IL-12 (BioLegend, 10 ng/mL), IL-18 (Invivogen, 50 ng/mL) and N-803 (ImmunityBio, 175 ng/mL) for 14-16 hours to generate M-CENK cells. A fixed final concentration of memory inducing cytokines IL-12 (10 ng/ml), IL-18 (50 ng/ml), and N-803 (175 ng/mL) were added to the culture as cytokine cocktail in fresh NK-XM media. This marks the beginning of a 14 (±2) hour cytokine cocktail treatment stage that induces a memory phenotype on the CD56 positive cells contained within the culture vessel. M-CENK cells were then harvested in 5% human albumin and diluted with CS-10 (at a ratio of 1:1) (“IB media’) (comprising 5% human albumin in Cryostorx CS-10 cell cryopreservation media) and cryopreserved.

For comparison, M-CENK cells were also created utilizing Apheresis methodology as described in previous disclosure (US2024/0132844). Primary NK cells were enriched from apheresis material and expanded in N-803-containing media, followed by stimulation with a cocktail containing N-803 (IL-15), IL-18, and IL-12, as disclosed above, to produce M-CENK cells).

### Cytotoxicity Assay:

Cytotoxicity assays can be performed using methods well known in the art. In one embodiment, the effector cells, for example, the MCENK cells disclosed herein, can be resuspended by up and down pipetting of the cell cultures. Cells viability can be determined by automated counting (NC-200 Nucleocounter. Target cells can be labelled with Calcein-AM dye, and dilutions of target and effector cells to the required cell concentrations can be made in suitable media. In some cases, the effector and target cells are mixed at different effector to target (E:T of 20:1, 10:1, 5:1, 2.5:1, 1.25:1, 0.62:1, 0.31:1, and 0.15:1) ratios in a 96-well plate and co-incubated for 4 h in a 5% CO2 atmosphere 37° C. incubator. Fluorescent Calcien released from the lysed cells in the culture supernatant is measured using plate reader. Amount of released Calcein is proportional to the percentage of target cell lysis.

### ADCC Assay:

Suspension-growing cell lines were resuspended by up and down pipetting of the cell cultures. Cells viability was determined by automated counting. Target cells were labelled with Calcein-AM dye, and dilutions of target and effector cells to the required cell concentrations were made in cell growth media. Target cells were pre-incubated with monoclonal antibodies (e.g., Rituxan) or no antibody for 30 min at R.T. The antibody is typically added to the reaction at a concentration of 0.5 μg/ml-10 μg/ml, for example, 1 μg/ml-5 μg/ml. Antibody-labelled target cells (and no-antibody controls) were then mixed with effector cells at different effector to target ratios (E:T of 20:1, 10:1, 5:1, 2.5:1, 1.25:1, 0.62:1, 0.31:1, and 0.15:1) in a 96-well plate and co-incubated for 4 h in a 5% CO2 atmosphere 37° C. incubator. Fluorescent Calcien released from the lysed cells in the culture supernatant is measured using plate reader. Amount of released Calcein is proportional to the percentage of target cell lysis.

### Exosome Isolation

Extracellular vesicles (exosomes and/or microvesicles) were isolated from the M-CENK cell culture comprising the M-CENK cells prepared as disclosed above by ultracentrifugation. The cell culture is centrifuged at 300×g for 10 minutes, and the cell pellet discarded. The supernatant is centrifuged at 2000×g for 20 minutes, and the pellet (cell debris) is discarded. The resulting supernatant is subjected to ultracentrifugation at 100,000×g for 80 minutes. The resulting pellet is washed with phosphate-buffered saline and subjected to ultracentrifugation at 100,000×g for 80 minutes. The washed pellet containing exosomes is retained for further studies.

The exosome preparations can be analyzed for the presence of several proteins, for example, Rab5B, perforin, Fas ligand (FasL), granzyme B, and granulysin, using methods well known in the art, for example, Western Blot. The cytotoxicity of the exosome preparations can be analyzied by incubating with target cells, for example, Jurkat cells, for a period of time (2 hours to 20 hours). Cytotoxicity can be determined by Prodidium iodide (PI) assay, and data can be expressed as the percent of cells that are PI positive (indicative of dead cells). Exemplary methods of isolation and analysis of the exosome are also described in U.S. Pat. No. 10,258,649.

### Lysates

To obtain M-CENK cell lysate, M-CENK cells are expanded at a density of 1×10⁶ cells/mL, harvested by centrifugation, and washed with phosphate-buffered saline (PBS). The cells were resuspended at 10⁸ cells/mL in a Balanced Salt Solution (BSS) cell lysis buffer (BSS: 250 mM NaCl, 50 mM Tris-HCl, pH 7.4) and lysed by either: a) 3 cycles of freeze (in liquid nitrogen for 30 seconds) and thaw (quick thaw for about 2 minutes in a 37° C. water bath) orb) 3 rounds of sonication at 20 kHz for 5 seconds each. No detergent is added to prevent proteins in the lysate from denaturing. Since the perforin/granzyme pathway is Ca++ dependent, no EDTA is added. The lysate is then centrifuged at 3,000 g, at 4° C., for 10 minutes to remove intact cells, insoluble cell debris and genomic DNA. The supernatant (the lysate) is collected and used either fresh or from cryopreserved (minus 80° C.) stock.

Flow cytometric/fluorescence-activated cell sorting (FACS) cytotoxicity assays can be used to assess the effect of the cell lysate on target cells, for example, the K562 cells. Target cells can be stained using the membrane dye PKH-67GL (Sigma-Aldrich®, St. Louis, MO) and incubated with M-CENK cell lysate for 30 min (Experiment 1) or 2 hrs (Experiment 2) at 37° C. Dead cells were stained with propidium iodide (PI) (Molecular Probes, Eugene, OR) and samples can be analyzed by flow cytometry. Percentage of dead cells can be determined by the percentage of PI positive cells within the PKH-67GL positive target cell population. % Killing was calculated as follows=[% dead target cells in sample−% spontaneous dead target cells]/[100−% spontaneous dead target cells].

### Results

M-CENK cells were enriched from apheresis material that consists of different immune populations, including NK cells, T cells, monocytes, B cells, and granulocytes.

It was tested if the depletion of a select population may influence the quality of M-CENK cells. Towards this goal, CD3+ T cells and CD14+ cells were removed from apheresis material before NK cell enrichment and M-CENK generation.

Immunoprofiling was performed on an apheresis product intermediate (described in previous disclosure (US2024/0132844) and on CD3− CD14− Cell Fraction from Apheresis product (collected using CliniMACS® Plus Process). As expected, an abundance of NK cells was observed in CD3− CD14− Cell Fraction (Table 2), and a minor fraction [of monocytes was observed in CD3− CD14− Cell fraction (using CliniMACS® Plus Process). It is noted that a CD3− CD14+ fraction was analyzed as well. 93% of cells were Monocytes. No PD process was conducted using CD3− CD14+ cells except freezing them for future use.

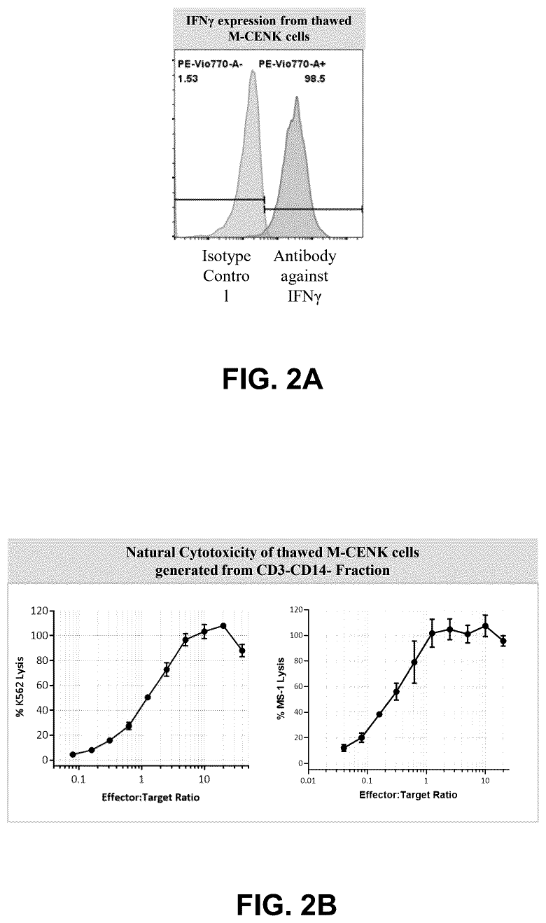

The desired cell population was removed from apheresis material using a column-based magnetic cell separation technique (for example, using column composed of ferromagnetic spheres, such as the ones available from Miltenyi). Depleting CD3+ and CD14+ cells changed the composition of apheresis material. An increased percentage of NK cells were present in the CD3− CD14− Apheresis material (Table 3 and FIG. 2).

TABLE 3
CyTOF-based analysis of Apheresis Material: Apheresis
Material was stained with MaxPar kit antibodies,
acquired on CyTOF, and the result in Table 3 shows
the percent composition of different cell types.

| | Source | |
|---|---|---|
| | Apheresis Material | |
| | Intermediate | CD3−CD14− Apheresis |
| Population | (Cryopreserved AMI) | Fraction |
| CD8 T Cells | 21.28 | 0 |
| CD4 T Cells | 31.39 | 0 |
| Treg | 0.84 | 0 |
| Gamma Delta T Cell | 2.83 | 0 |
| MAIT & NKT | 1.15 | 0 |
| B Cells | 6.42 | 22.37 |
| NK Cells | 7.86 | 47.51 |
| Monocytes | 20.1 | 8.64 |
| Dendritic Cells | 1.13 | 4.85 |
| Granulocytes | 1.7 | 6.19 |

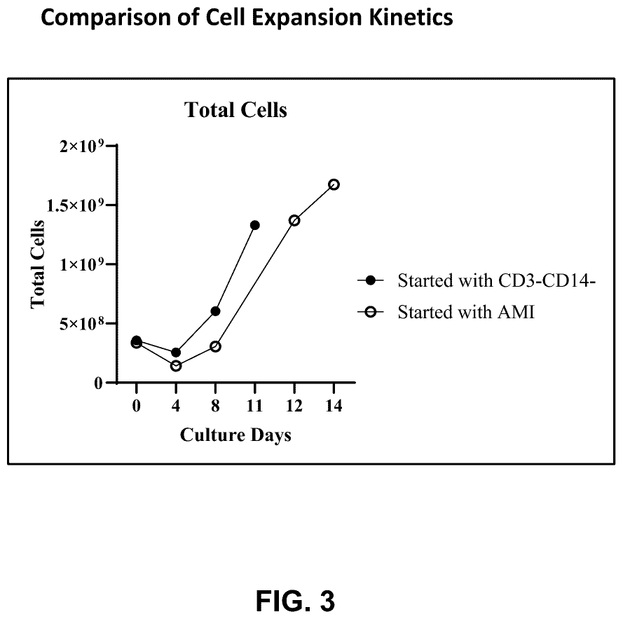

A faster NK cell enrichment, expansion and kinetics (FIG. 3) was observed in culture derived from CD3− CD14− Apheresis material compared to culture derived from thawed Apheresis Material (Table 4).

TABLE 4
NK Cell Enrichment Kinetics: Culture initiated from AMI (as
disclosed in US Pat. Pub. No. 2024/0132844A1) vs CD3−CD14−
apheresis fraction (i.e., CD3−/CD14− double depleted)
was monitored for CD56+ NK cell enrichment by flow cytometry.

Time to achieve purity

| | Apheresis Material | |
|---|---|---|
| | Intermediate (Cryo | CD3− CD14− |
| Population | AMI) | Apheresis Fraction |
| % CD56+NK Cells | 82% CD56+ cells | 95% CD56+ cells |
| | in 14 days | in 8 days |

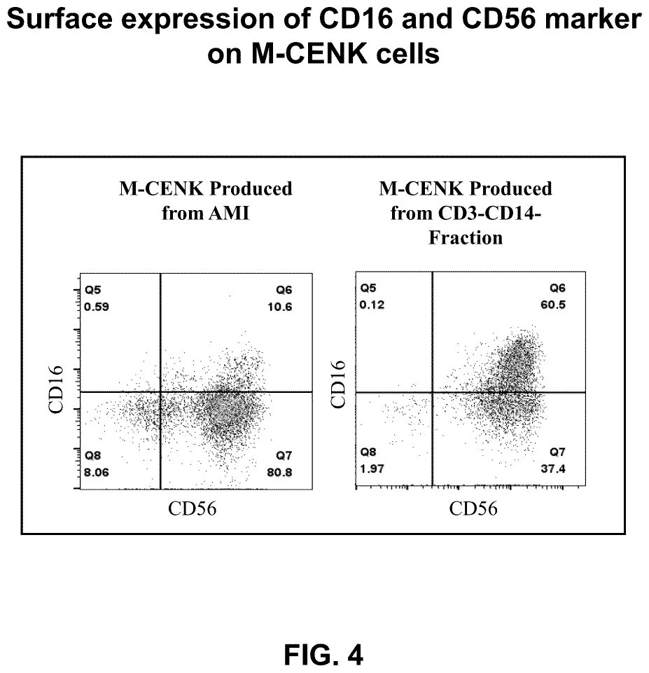

M-CENK cells generated from apheresis material using the GEN-3 Process (i.e., a conventional method) showed reduced expression of the CD16 receptor. In contrast, M-CENK generated using the CD3− CD14− Apheresis product maintained high expression of the CD16 receptor (FIG. 4).

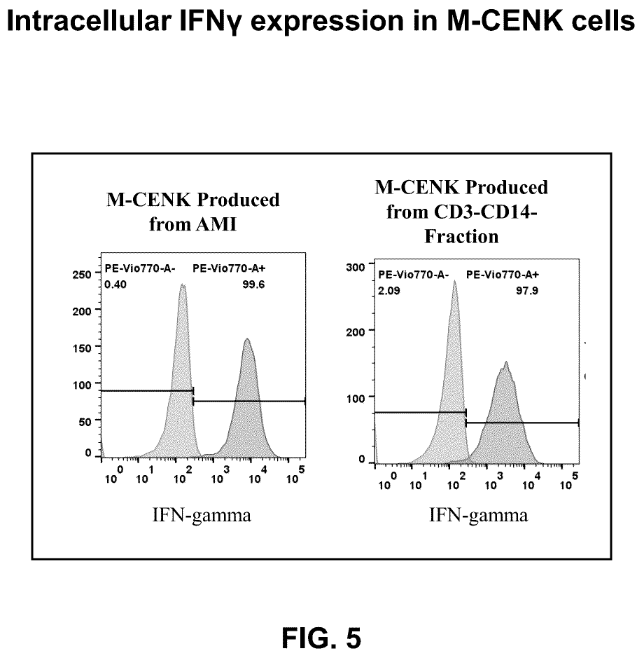

Different killing activities of M-CENK cells were tested to evaluate if higher CD16 expression influence the M-CENK cell attributes. M-CENKcells generated from Apheresis Material or the CD3− CD14− Apheresis fraction showed comparable IFN-gamma expression on CD56+ cells. (FIG. 5).

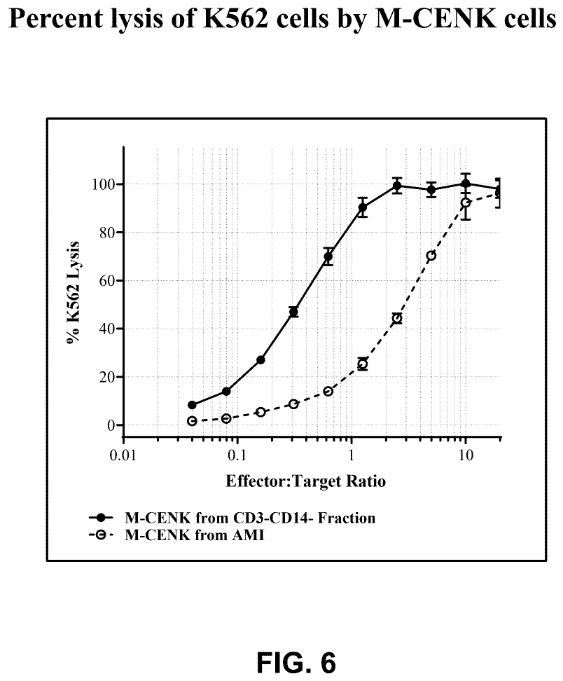

To compare direct cytotoxicity, M-CENK cells from different sources were incubated with NK-sensitive K562 cells and the cytotoxicity assays were performed as disclosed above. Although M-CENK cells from both sources killed K562 cells, M-CENK cells from the CD3− CD14− Apheresis fraction killed the target cells with faster kinetics (FIG. 6), as indicated by a ten-fold decrease in effector:target ratio.

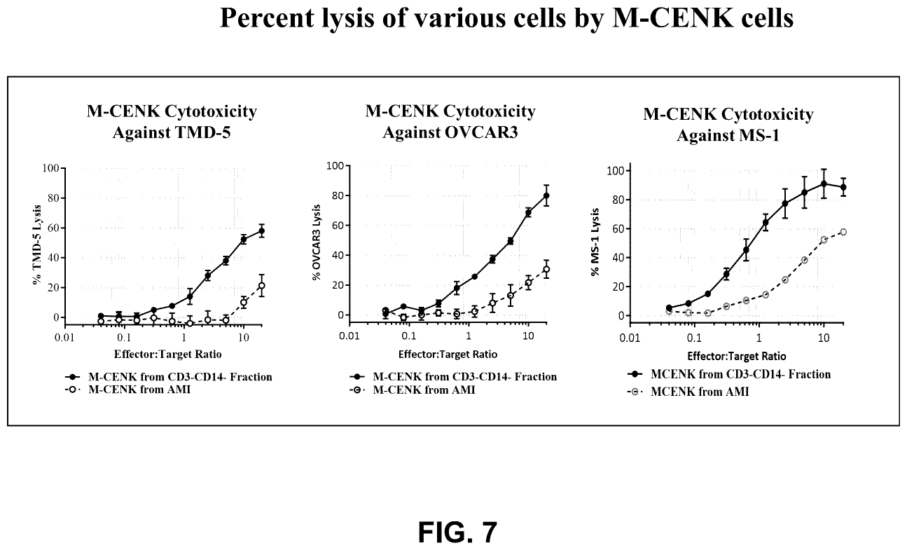

To compare cytotoxicity against NK-resistant cells, M-CENK cells from different sources were incubated with tumor cells like MS-1, TMD-2, and OVCAR3, and the cytotoxicity assays were performed as disclosed above. M-CENK cells from CD3− CD14− Apheresis fraction showed superior killing of all the tested tumor cells when compared to M-CENK cells from apheresis material (FIG. 7).

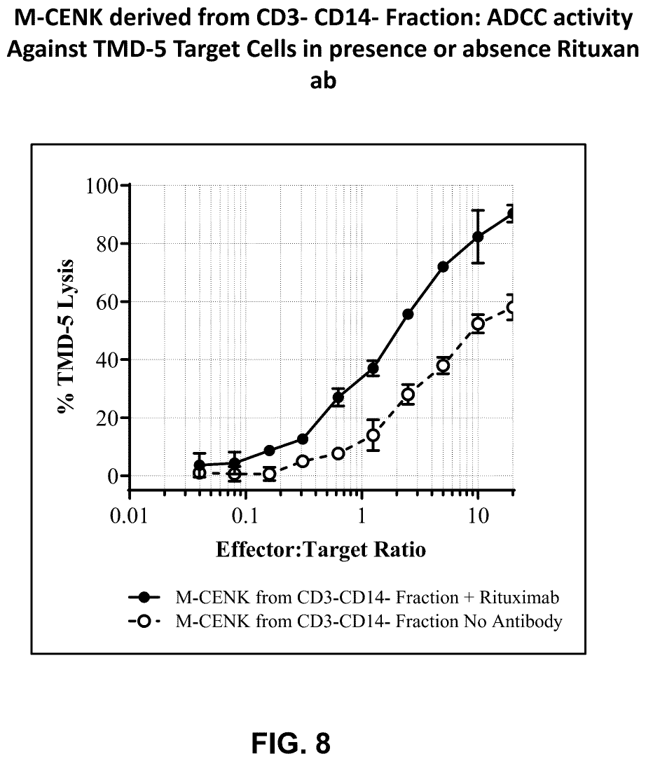

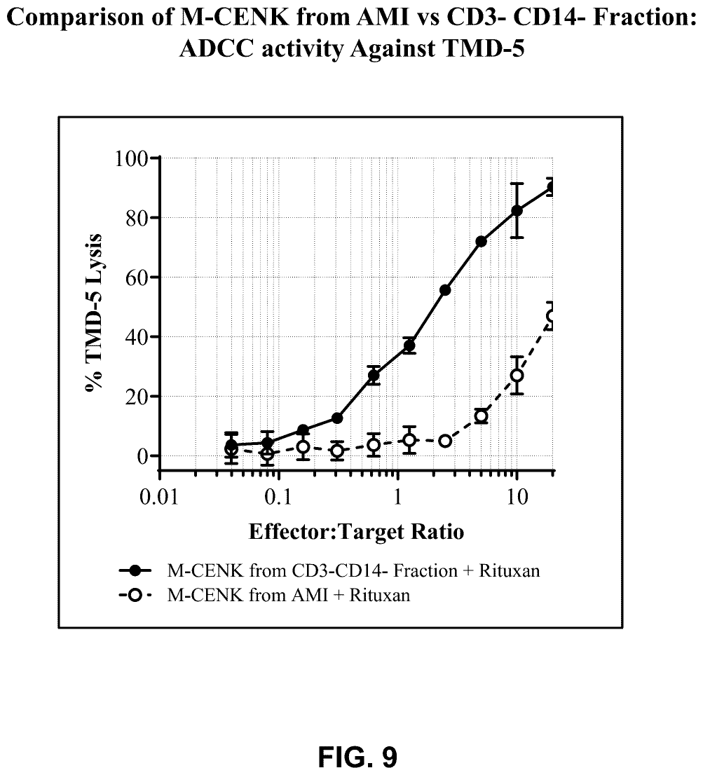

To evaluate the ADCC activity of M-CENK cells from CD3− CD14− Apheresis fraction, cells were incubated with TMD-5 in the presence/absence of Rituxan® antibody (FIG. 8). To compare the ADCC activity of M-CENK cells generated from two different sources, cells were incubated with TMD-2 in the presence of Rituxan® antibody (FIG. 9). The ADCC assays were performed as disclosed above.

As shown in the present example, there was efficient enrichment of CD56+ cells from a CD3− CD14− double-depleted fraction of an Apheresis product purified using CliniMACS® plus. NK and B cells were abundant in CD3− CD14− cell fraction, as evident from the CyTOF data. M-CENK cells showed relatively higher expression of CD16 compared to the control. Thawed M-CENK from AMI using the process described in US Pat. Pub. No. 2024/0132844A1 showed less ADCC against OVCAR3 and TMD-5; (though similar Cytotoxicity observed against MS-1 and K562 target cells). These cumulative data may suggest, NK cells enriched and expanded from CD3− CD14− cells are different and unique M-CENK cells though generated from same Apheresis product.

### Example 3: Immune Profiling of M-CENK Cell Population

M-CENK cell population obtained using the method described in Examples 1 and 2 were stained with a panel of 30 antibody (Maxpar® Direct™ Immune Profiling Assay™, 30 Marker, Standard BioTool). The stained cells were analyzed using Helios system, a CyTOF (Cytometry by Time Of Flight) platform. The results were analyzed using Maxpar Pathsetter software and summarized in Table 5.

TABLE 5
Immune profiling of the M-CENK cell population

| Cell Types | Percent of live cells |
|---|---|
| CD8+ T cells | 0.01 |
| CD4+ T cells | 0.00 |
| Treg cells | 0.00 |
| Th1-like cells | 0.00 |
| Th2-like cells | 0.00 |
| Th17-like cells | 0.00 |
| γδT cells | 0.00 |
| MAIT, NKT cells | 0.00 |
| B cells | 0.02 |
| NK cells | 98.16 |
| Monocytes | 0.00 |
| Dendritic cells | 0.01 |
| Granulocytes | 0.21 |

The results above demonstrate that shows that M-CENK cell population generated from CD3− CD14− Fraction of the blood cells contained a high percentage of NK cells. In this illustrative example, the percentage of NK cells in the M-CENK cell population were 98.16%.

### Example 4: Characterization of Surface and Intracellular Markers for M-CENK Cell Population

The M-CENK cell population obtained using the methods disclosed in Examples 1 and 2 were stained with a panel of 24 antibodies which recognize various surface markers and intracellular markers. Cells were stained with surface markers first followed by fixing and intracellular staining. Once cells were stained, they were loaded to the Helios system, a CyTOF (Cytometry by Time of Flight) platform. The results were analyzed using the FCS Express software and summarized in Table 6.

TABLE 6
Extended Characterization of Surface and Intracellular Markers of
M-CENK Drug product generated from CD3−CD14− Fraction.

| Markers | | | % expression |
|---|---|---|---|
| Surface | Activation | NKp30+ | 95.4 |
| Marker | Marker | NKp46+ | 74.2 |
| | | NKG2D+ | 52.7 |
| | | CD16+ | 3.3 |
| | | CD25+ | 96.5 |
| | Inhibitory | NKG2A+ | 99.2 |
| | Marker | KIR3DL1+ | 44.0 |
| | | TIGIT+ | 18.3 |
| | Additional | CD57+ | 17.1 |
| | Marker | CD69+ | 99.8 |
| | | HLA-DR+ | 99.8 |
| | | CD38+ | 89.3 |
| | | CD27+ | 3.9 |
| | | CD62L+ | 17.2 |
| | Stress | PD-1+ | 4.6 |
| | Marker | LAG-3+ | 14.1 |

| Intracellular Marker | Perforin+ | 94.9 |
|---|---|---|
| | Granzyme B+ | 99.5 |
| | Ki-67+ | 78.7 |
| | IFNγ+ | 98.1 |

### Example 5. Cytokine Production and Cytotoxicity of the M-CENK Cell Population

M-CENK cells are a major source of Interferon gamma (IFN-γ). To analyze the IFN-γ production, the M-CENK cell population obtained according to methods described in Examples 1 and 2 were cryopreserved and then thawed. Intracellular IFN-γ staining was performed on the thawed cells, and the stained cells were loaded to a MACSQuant® analyzer10. Data files were analyzed to determine the mean fluorescence intensity (MFI) for IFN-γ in CD56+ cells. The results showing the presence of IFNγ in thawed M-CENK are shown in FIG. 2A. By combining surface staining (as shown in Example 4) and intracellular cytokine staining, the percentage of IFN-γ producing CD56+ cells can be identified.

The activity of the thawed M-CENK cells were measured by cytotoxicity against cells from two tumor cell lines: K562 and MS-1. The cytotoxicity of the cells was measured using Calcein-AM based cytotoxicity assay, in which these M-CENK cells cause lysis (cell death) of target tumor cells. As the cells get lysed, they release Calcein-AM, which was measured.

K562 is a human leukemia cell line and is susceptible to NK cells. MS-1 is a skin carcinoma cells with a resistance to general NK cell cytotoxicity. Thawed M-CENK cells were mixed with K562 cells and MS-1 cells, respectively, at various ratios (E:T ratio. E, represents Effector cells and T, represents target cells) in the assay. Release of Calcein-AM was measured using Perkin Elmer Victor X5 Plate Reader and data are analyzed using software (Excel, Prism or equivalent). The results are shown in FIG. 2B. The results indicate that the M-CENK cells produced possess the natural cytotoxicity of the NK cells, i.e., they can kill target cells without prior activation by antigen. (Vivier, E., et al., Science. 2011 Jan. 7; 331(6013): 44-49; Hudspeth, K., et al., Front Immunol. 2013 Mar. 19, Vol. 4; and Barrow, A. D. et. al., Frontiers in Immunology, May 2019, Vol 10, Article 909).

1. A method of generating memory-like cytokine enhanced natural killer (M-CENK) cells from a blood sample, the method comprising:
(a) removing CD3+ cells and CD14+ cells from the blood sample to obtain a CD3− CD14− population of cells; and
(b) incubating the CD3− CD14− population of cells with a first cytokine composition comprising a stabilized IL-15 analog or fusion protein comprised thereof for a first period (optionally 8-10 days), and
(c) incubating the CD3− CD14− population of cells with a second cytokine composition comprising i) IL-15 or a stabilized derivative thereof, ii) IL-18 or a stabilized derivative thereof, and iii) IL-12 or a stabilized derivative thereof for a second period (optionally 14-16 hours), thereby producing a population of CD56+ enriched M-CENK cells.

2. The method of claim 1, wherein the IL-15 or stabilized derivative thereof is in a concentration ranging from 25 to 175 ng/ml.

3. The method of claim 1, wherein the IL-18 or stabilized derivative thereof is in a concentration ranging from 25 to 50 ng/ml.

4. The method of claim 1, wherein the IL-12 or stabilized derivative thereof is in a concentration ranging from 5 to 10 ng/ml.

5. The method of claim 1, wherein the blood sample is obtained from a cancer patient.

6. The method of claim 5, wherein a cancer of the cancer patient is a small cell lung cancer, an ovarian cancer, a breast cancer, a leukemia, a myelogenous leukemia, or an acute lymphoblastic leukemia.

7. The method of claim 1, wherein step (a) is by magnetic cell separation.

8. The method of claim 1, wherein the first period lasts 7 to 12 days.

9. The population of CD56+ enriched M-CENK cells produced from the method of claim 1.

10. The population of CD56+ enriched M-CENK cells of claim 9, wherein greater than 90% of the cells are CD56+.

11. The population of CD56+ enriched M-CENK cells of claim 9, wherein greater than 60% of the cells are CD16+.

12. A method of treating a patient diagnosed with cancer, the method comprising:
(a) obtaining a blood sample from the patient;
(b) removing CD3+ cells and CD14+ cells from the blood sample to obtain a CD3− CD14− fraction;
(c) contacting the CD3− CD14− fraction with IL-15 or a stabilized derivative thereof for a first period of time;
(d) contacting the CD3− CD14− fraction with IL-12 or a derivative thereof, IL-18 or a derivative thereof, and IL-15 or a derivative thereof, for a second period of time, thereby obtaining a cell culture comprising a population of CD56+ enriched M-CENK cells;
(e) centrifuging the cell culture to produce a supernatant;
(f) isolating exosomes from the supernatant; and
(g) administering the isolated exosomes to the patient, thereby treating the cancer.

13. A method of treating a patient diagnosed with cancer, the method comprising administering a therapeutically effective amount of the CD56+ enriched M-CENK cells of claim 9 to the patient.

14. The method of claim 12, wherein the cancer is a solid tumor, optionally, the solid tumor is a metastatic solid tumor.

15. The method of claim 12, wherein the cancer is a small cell lung cancer, an ovarian cancer, a breast cancer, a leukemia, a myelogenous leukemia, or a acute lymphoblastic leukemia.

16. The method of claim 12, wherein greater than 90% of the CD56+ enriched population of cells are CD56+.

17. The method of claim 12, wherein greater than 60% of the CD56+ enriched population of cells are CD16+.

18. The method of claim 12, wherein the method further comprises administering an antibody.

19. The method of claim 18, wherein the antibody is an anti-CD20 antibody.

20. A kit for preparing CD56+M-CENK cell exosomes, the kit comprising: i) reagents for depleting CD3+ cells and CD14+ cells from a blood sample; ii) IL-15 or a fusion protein comprised thereof, iii) IL-12 or a fusion protein comprised thereof, and iv) IL-18 or a fusion protein comprised thereof.

21. A population of CD56+ enriched M-CENK cells wherein at least 90% of the cells are CD3−, CD14−, and CD56+, and wherein at least 60% of the cells express CD16.

22. The population of CD56+ enriched M-CENK cells of claim 21, wherein the cells are frozen and thawed.

23. A cell culture mixture comprising a population of blood cells that are CD3− CD14−, a first cytokine composition comprising a stabilized IL-15 analog or fusion protein.

24. The cell culture mixture of claim 23, further comprising a second cytokine composition comprising one or more of i) IL-15 or a stabilized derivative thereof, ii) IL-18 or a stabilized derivative thereof, and iii) IL-12 or a stabilized derivative thereof.
